(19) United States
(12) Patent Application Publication
(10) Pub. No.: US 2025/0130220 A1
(43) Pub. Date: Apr. 24, 2025

(54) LYMPHOCYTE POTENCY ASSAY

(71) Applicant: KSQ Therapeutics, Inc., Lexington, MA (US)

(72) Inventors: Micah Benson, Lexington, MA (US); Michael R. Schlabach, Lexington, MA (US); Karrie Wong, Lexington, MA (US)

(73) Assignee: KSQ Therapeutics, Inc., Lexington, MA (US)

(21) Appl. No.: 18/689,947

(22) PCT Filed: Sep. 7, 2022
(86) PCT No.: PCT/US2022/076028

§ 371 Date: Mar. 7, 2024

Related U.S. Application Data
(60) Provisional application No. 63/241,768, filed on Sep. 8, 2021.
(60) Provisional application No. 63/291,655, filed on Dec. 20, 2021.
(60) Provisional application No. 63/391,118, filed on Jul. 21, 2022.

(51) Int. Cl. G01N 33/50

(52) U.S. Cl. CPC G01N 33/5011; G01N 33/505

(57) ABSTRACT

The present disclosure provides methods and materials useful for measuring the antitumor potency of lymphocytes, such as tumor infiltrating lymphocytes.

### RELATED APPLICATIONS

This application claims the benefit under 35 U.S.C. § 119(e) of U.S. provisional application No. 63/241,768, filed Sep. 8, 2021, U.S. provisional application No. 63/291,655, filed Dec. 20, 2021, and U.S. provisional application No. 63/391,118, filed Jul. 21, 2021, each of which is incorporated by reference herein in its entirety.

### REFERENCE TO AN ELECTRONIC SEQUENCE LISTING

The contents of the electronic sequence listing (K071370018WO00-SEQ-HJD.xml; Size: 21,568 bytes; and Date of Creation: Sep. 7, 2022) is herein incorporated by reference in its entirety.

### FIELD

The present disclosure relates to methods and compositions useful for assessing the antitumor potency of lymphocytes, such as tumor infiltrating lymphocytes.

### BACKGROUND

Lymphocytes are white blood cells essential to the immune system. Tumor-infiltrating lymphocytes (TILs) are white blood cells, including T cells and B cells, that have left the bloodstream and migrated toward a tumor. The presence of lymphocytes in tumors is often associated with better clinical outcomes, and indeed, lymphocytes such as TILs have been implicated in killing tumor cells. Lymphocytes are routinely used as an adoptive cell therapy (ACT) to treat certain types of cancer. The adoptive transfer of TILs, for example, is a powerful approach to the treatment of bulky, refractory cancers, especially in patients with poor prognoses. In ACT, the cells are expanded ex vivo and must be characterized for potency prior to being infused back into patients.

TIL potency assays, which measure direct or indirect biological activity specifically relevant to TILs, are especially important for ACT using TILs given the broad heterogeneity of TIL/tumor specificity among patients. Additionally, TIL potency assays are required by the U.S. Food and Drug Administration (FDA) to ensure quality of individual TIL products that may be used in ACT.

Importantly, not all TIL potency assays are considered biologically relevant, either by the biological context of the assay, or the assay endpoint. Increasing regulatory guidelines require that TIL potency assay read-outs must correlate with in vivo function (e.g., tumor recognition and cell death) in order to translate to clinical efficacy. Although assays may use non-cell based TIL stimulation approaches and be based on T cell properties that are surrogates for cytotoxicity (e.g., the use of interferon-gamma (IFN-γ) release assay for an assessment of TIL potency), in vitro TIL potency assays based on tumor cell-mediated activation of TILs yield a more accurate representation of TIL potency and, when cytotoxicity or surrogate endpoints including IFN-γ release are used, are considered in the field to be most evident of correlation with clinical efficacy (see, e.g., de Wolf et al. Cytotherapy 2018 May; 20(5):601-622). Such biologically relevant TIL potency assays are currently limited in the field.

### SUMMARY

The present disclosure provides lymphocyte potency assays, such as TIL potency assays (also referred to herein as TIL antitumor potency assays), that may be used to assess the ability of lymphocytes to generate a clinically-relevant antitumor response. In some embodiments, the present disclosure provides immortalized cells (e.g., tumor cells) that comprise a molecule that activates lymphocytes, such as a molecule that activates T cells (e.g., a molecule that binds to a T cell antigen). This interaction between the immortalized cells, such as tumor cells, and the lymphocytes can be used to assess the potency of the lymphocytes as an antitumor therapy for cancer, for example. Prior to the assays described herein, the majority of lymphocyte (e.g., TIL) potency assays were based on non-cell-based lymphocyte activation and cytokine (e.g., IFN-γ) release assays as a measure of lymphocyte activity and could only be regarded as a physiologically-irrelevant stimulation for interrogation of cytolytic function.

The present disclosure provides data showing that tumor cells, such as human melanoma A375 cells, engineered to express membrane associated anti-CD3 (OKT3) antibody, for example, can be used to activate TIL antitumor function. OKT3 is an activating antibody used to activate lymphocytes, either in its soluble form or bound to beads and tethered to magnetic beads. Unexpectedly, the process of decreasing the affinity of A375-pKSQ367 cells for TILs by incorporating mutations into the OKT3 antibody that decreases the affinity of the membrane-associated binding domain for TILs serves to improve the usefulness of the assay for evaluating TIL potency, for example, in three-dimensional in vitro tumor spheroid functional assays.

The present disclosure also provides data showing that, in some embodiments, two-dimensional monolayer cell cultures are better suited for assessing antitumor activities of lymphocytes, while three-dimensional multilayer spheroid cultures, in some embodiments, are better suited for assessing functional properties of different populations of lymphocytes, such as edited TILs versus unedited TILs. The spheroid setting described herein enabled the identification and assessment of multiple aspects of TIL function/biology, including cytotoxicity, cytokine production, proliferation, and phenotypic changes. On the target side, the spheroid dimension offers a higher level of cell-cell contact and interactions that is closer to that occurring in vivo. Without being bound by theory, the potency assays provided herein offer elegant yet complex cellular microenvironments with which to compare and contrast changes in morphological as well as other cellular properties.

Aspects of the present disclosure provide methods for assessing potency of lymphocytes (e.g., T cells), comprising: coculturing lymphocytes (e.g., T cells) and immortalized cells, wherein the immortalized cells comprise a molecule that activates a lymphocyte (e.g., T cell), and assessing potency of the lymphocytes. In some embodiments, the lymphocytes are non-engineered (e.g., do not include non-naturally-occurring genomic modification).

Other aspects of the present disclosure provide methods for assessing potency of TILs, comprising: coculturing TILs and engineered tumor cells, wherein the engineered tumor cells comprise a molecule that activates a T cell, and assessing potency of the TILs.

Yet other aspects of the present disclosure a method for assessing potency of polyclonal T cells, comprising coculturing polyclonal T cells and immortalized cells, wherein the immortalized cells comprise a molecule that activates a T cell, and assessing potency of the polyclonal T cells.

In some embodiments, the molecule binds to a T cell antigen. In some embodiments, the TILs express the T cell antigen. In some embodiments, the polyclonal T cells express the T cell antigen. In some embodiments, the T cell antigen is a CD3 antigen. In some embodiments, the immortalized cells express the molecule.

In some embodiments, the molecule is an antibody or an antibody fragment. For example, the antibody fragment may be selected from a single-chain variable fragment (scFv), a F(ab′)2 fragment, a Fab fragment, a Fab′ fragment, and an Fv fragment. In some embodiments, the antibody fragment is an scFv. In some embodiments, the antibody or antibody fragment is respectively an OKT3 antibody or OKT3 antibody fragment. In some embodiments, the OKT3 antibody fragment is a membrane-bound OKT3 (mOKT3) scFv. For example, the mOKT3 scFv may be a low-affinity mOKT3 scFv variant.

In some embodiments, the molecule binds to CD3 with a dissociation constant (KD) that is lower than the KD of mOKT3 scFv, wherein the KD of mOKT3 scFv is about 5×10⁻¹⁰ M. In some embodiments, the low-affinity mOKT3 scFv variant comprises an amino acid sequence having R55 and Y57 mutations, relative to the amino acid sequence of SEQ ID NO: 2. In some embodiments, the low-affinity mOKT3 scFv variant comprises an amino acid sequence having R55M and Y57A mutations, relative to the amino acid sequence of SEQ ID NO: 2. In some embodiments, the low-affinity mOKT3 scFv variant binds to CD3 with a dissociation constant KD that is least 250-fold lower than the KD of mOKT3 scFv. In some embodiments, the low-affinity mOKT3 scFv variant comprises an amino acid sequence having R55L and Y57T mutations, relative to the amino acid sequence of SEQ ID NO: 2. In some embodiments, the low-affinity mOKT3 scFv variant binds to CD3 with a dissociation constant KD that is least 1000-fold lower than the KD of mOKT3 scFv.

In some embodiments, the molecule is selected from phytohaemagglutinin (PHA) and concanavalin A (ConA). In some embodiments, the molecule is a bacterial superantigen, for example, staphylococcal enterotoxin B (SEB). In some embodiments, the molecule is a membrane-tethered molecule.

In some embodiments, the TILs are engineered TILs (eTILs). In some embodiments, the eTILs are edited eTILs. In some embodiments, the edited eTILs comprise a genomic modification.

In some embodiments, the polyclonal T cells comprise neoantigen-specific T cells.

In some embodiments, the polyclonal T cells are from peripheral blood.

In some embodiments, the polyclonal T cells are from bone marrow.

In some embodiments, the immortalized cells comprise a clonal population of immortalized cells. In some embodiments, the immortalized cells are immortalized human cells. In some embodiments, the immortalized cells are engineered (e.g., human) cancer cells. In some embodiments, the engineered cancer cells are selected from engineered melanoma cells, engineered colorectal cancer cells, engineered bile duct cancer cells, and engineered breast cancer cells.

In some embodiments, the coculturing is for at least 4 hours (e.g., about 4-6, about 4-8, about 4-12 hours, about 4-18 hours, or about 4-24 hours). In some embodiments, the coculturing is for at least 12 hours. In some embodiments, the coculturing is for at least 24 hours, at least 48 hours, or at least 72 hours. In some embodiments, the coculturing is for about 1-days, about 1-3 days, about 1-4 days, about 1-5 days, about 1-6 days, or about 1-7 days.

In some embodiments, the assessing potency comprises measuring surrogate markers of TIL reactivity to tumor, including release of effector cytokines such as IFN-γ or expression of CD107a. In some embodiments, the assessing potency comprises measuring growth of the immortalized cells. In some embodiments, the assessing potency comprises measuring cell death and/or viability of the immortalized cells.

In some embodiments, the measuring comprises performing a cell viability assay. In some embodiments, the measuring comprises performing a cell cytotoxicity assay. In some embodiments, the measuring comprises performing an assay selected from real-time cell viability assays, ATP cell viability assays, live cell protease viability assays, tetrazolium reduction cell viability assays, resazurin reduction cell viability assays, dead-cell protease release cytotoxicity assays, lactate dehydrogenase release cytotoxicity assays, and DNA dye cytotoxicity assays.

Further aspects of the present disclosure provide methods for assessing potency of tumor infiltrating lymphocytes (TILs), comprising: coculturing TILs and a clonal population of engineered cancer cells, wherein the engineered cancer cells express an anti-CD3 antibody or anti-CD3 antibody fragment; and assessing death and/or viability of the engineered cancer cells.

In some embodiments, the TILs express a CD3 antigen.

Still other aspects of the present disclosure provide methods for assessing potency of polyclonal T cells, comprising: coculturing polyclonal T cells and a clonal population of engineered cancer cells, wherein the engineered cancer cells express an anti-CD3 antibody or anti-CD3 antibody fragment; and assessing death and/or viability of the engineered cancer cells.

In some embodiments, the polyclonal T cells express a CD3 antigen.

In some embodiments, the engineered cancer cells express an anti-CD3 antibody fragment. For example, the anti-CD3 antibody fragment may be an anti-CD3 single-chain variable fragment (scFv). In some embodiments, the anti-CD3 scFv is mOKT3 scFv. In some embodiments, the mOKT3 scFv is a low-affinity mOKT3 scFv variant.

In some embodiments, the low-affinity mOKT3 scFv variant binds to CD3 with a dissociation constant (KD) that is lower than the KD of mOKT3 scFv, wherein the KD of mOKT3 scFv is about 5×10⁻¹⁰ M. In some embodiments, the low-affinity mOKT3 scFv variant binds to CD3 with a dissociation constant KD that is least 1000-fold lower than the KD of mOKT3 scFv. In some embodiments, the low-affinity mOKT3 scFv variant comprises an amino acid sequence having R55L and Y57T mutations, relative to the amino acid sequence of SEQ ID NO: 2.

In some embodiments, the anti-CD3 antibody or anti-CD3 antibody fragment is respectively a membrane-tethered anti-CD3 antibody or membrane-tethered anti-CD3 antibody fragment.

In some embodiments, the TILs are engineered TILs (eTILs). In some embodiments, the eTILs are edited eTILs. In some embodiments, the edited eTILs comprise a genomic modification.

In some embodiments, the engineered cancer cells are selected from engineered melanoma cells, engineered colorectal cancer cells, engineered bile duct cancer cells, and engineered breast cancer cells. In some embodiments, the engineered cancer cells are engineered melanoma cells.

In some embodiments, the coculturing is for at least 24 hours, for example, about 24 to 72 hours.

In some embodiments, the measuring comprises performing an assay selected from real-time cell viability assays, ATP cell viability assays, live cell protease viability assays, tetrazolium reduction cell viability assays, resazurin reduction cell viability assays, dead-cell protease release cytotoxicity assays, lactate dehydrogenase release cytotoxicity assays, and DNA dye cytotoxicity assays.

Further aspects of the present disclosure provide methods for assessing potency of TILs, comprising: coculturing TILs, immortalized cells, and a bispecific molecule that activates a T cell and binds to the immortalized cells, and assessing potency of the TILs. In some embodiments, the TILs express a CD3 antigen. In some embodiments, the bispecific molecule comprises a molecule that binds CD3.

In some embodiments, the TILs are engineered TILs (eTILs). In some embodiments, the eTILs are edited eTILs. In some embodiments, the edited eTILs comprise a genomic modification.

Other aspects of the present disclosure provide methods for assessing potency of polyclonal T cells, comprising: coculturing polyclonal T cells, immortalized cells, and a bispecific molecule that activates a T cell and binds to the immortalized cells, and assessing potency of the polyclonal T cells. In some embodiments, the polyclonal T cells express a CD3 antigen. In some embodiments, the bispecific molecule comprises a molecule that binds CD3.

In some embodiments, the immortalized cells comprise a clonal population of immortalized cells. In some embodiments, the immortalized cells are human cells. In some embodiments, the immortalized cells are (e.g., human) cancer cells. In some embodiments, the cancer cells are selected from melanoma cells, colorectal cancer cells, bile duct cancer cells, and breast cancer cells.

In some embodiments, the bispecific molecule comprises a molecule that binds CD19. In some embodiments, the bispecific molecule comprises a molecule that binds CD3. In some embodiments, the bispecific molecule comprises a molecule that binds CD19 and CD3.

In some embodiments, the bispecific molecule is a CD19-CD3 BiTE®.

In some embodiments, the coculturing is for at least 24 hours, for example, about 24 to 72 hours.

In some embodiments, the measuring comprises performing a cell viability assay. In some embodiments, the measuring comprises performing a cell toxicity assay. In some embodiments, the measuring comprises performing an assay selected from real-time cell viability assays, ATP cell viability assays, live cell protease viability assays, tetrazolium reduction cell viability assays, resazurin reduction cell viability assays, dead-cell protease release cytotoxicity assays, lactate dehydrogenase release cytotoxicity assays, and DNA dye cytotoxicity assays.

### BRIEF DESCRIPTION OF THE DRAWINGS

The foregoing and other features and advantages of the present disclosure will be more fully understood from the following detailed description of illustrative embodiments taken in conjunction with the accompanying drawings.

FIG. 1 shows that A375 cells transduced with 0 μL, 25 μL, 50 μL, 100 μL, 200 μL, 400 μL, or 800 μL of pKSQ366 or pKSQ367 lentivirus encoding mOKT3 were cocultured with Human Pan-CD3⁺ T cells overnight. The following day, CD69 activation was assessed via flow cytometry. A375 cells transduced with varying amounts of pKSQ366 virus were found to lead to similar levels of CD69 activation, apart from 25 μL of virus. A375 cells transduced with pKSQ367 lead to slightly higher levels of CD69 activation and similar levels of activation were found with the varying amounts of virus used.

FIG. 2 shows that A375-pKSQ367 and non-transduced cells were stained with a goat-anti-mouse antibody to confirm mOKT3 surface expression. 99.2% of A375-pKSQ367 were found to be expressing mOKT3.

FIGS. 3A-3C show pre-REP TIL cocultured with A375-pKSQ367 for 3 days at varying effector cell to target cell ratios (E:T). No killing of A375 by pre-REP TIL is observed except for at 10:1 with D2777. Increased TIL recognition and subsequent killing of A375-pKSQ367 is observed at all E:T.

FIG. 4 shows that constructs with varying membrane anchors, signal peptides, and scFv linkers all produce robust activation of T cells. K562 cells with the indicated construct were cocultured with pan T cells. After 24 hours, T cells were stained for CD8 and CD69 surface expression and measured by flow cytometry. CD69 expression was high after coculture with K562 cells expressing mOKT3, regardless of the anchor used to tether it to the plasma membrane of the K562 cells.

FIG. 5 shows that high affinity A375 pKSQ367 shows high expression of mOKT3 marker in comparison to the A375 parental cell line from which it was derived.

FIG. 6 shows that low affinity line A375 pKSQ397 shows high mOKT3 marker expression. Increasing the volume of virus used in the transduction does not increase the expression of mOKT3 on the surface of these cells.

FIG. 7 shows that low affinity line A375 pKSQ398 shows medium levels of mOKT3 marker expression. Increasing the volume of virus used in the transduction does not increase the expression of mOKT3 on the surface of these cells.

FIG. 8 shows that after coculture with A375 pKSQ367 target cells, pan T cells show increased expression of CD69 on the cell surface. This indicates recognition of target cells (A375 pSKQ367) and activation of the pan T cells. In contrast, pan T cells show very little CD69 expression after coculture with A375 parental cells. This demonstrates a lack of recognition and therefore no increase in activation of the pan T cells. The pan T cells cultured alone as a control indicate the lack of basal CD69 expression.

FIG. 9 shows that after coculture with A375 pKSQ396 target cells, pan T cells show little expression of CD69 on the cell surface. This level of expression is comparable to pan T cells cocultured with A375 parental cells (FIG. 8). This demonstrates a lack of target cell recognition by the pan T cells and therefore no increase in their activation.

FIG. 10 shows that after coculture with A375 pKSQ397 target cells, pan T cells show expression of CD69 on the cell surface. This level of expression indicates recognition of target cells (A375 pSKQ397) and activation of the pan T cells. Target cells transduced with higher volumes of virus appear to have the ability slightly increase the activation of the pan T cells.

FIG. 11 shows that after coculture with A375 pKSQ398 target cells, pan T cells show expression of CD69 on the cell surface. This level of expression indicates recognition of target cells (A375 pSKQ398) and activation of the pan T cells. Target cells transduced with higher volumes of virus appear to have the ability slightly increase the activation of the pan T cells.

FIG. 12 shows that A375 parental cells were cocultured as a monolayer with gene target-edited TIL (No EP, OLFR, SOCS1, CBLB) from TIL donor 3239. Due to the lack of mOKT3 surface expression, no reduction, or differences in growth of target A375 cells was observed.

FIG. 13 shows that A375 pKSQ367 cells were cocultured as a monolayer with gene target-edited TIL (No EP, OLFR, SOCS1, CBLB) from TIL donor 3239. Due to the high levels of mOKT3 surface expression observed previously, a large reduction in growth of the target A375 pKSQ367 cells was demonstrated.

FIG. 14 shows that A375 pKSQ398 cells were cocultured as a monolayer with gene target-edited TIL (No EP, OLFR, SOCS1, CBLB) from TIL donor 3239. Because pKSQ398 has reduced affinity towards CD3 compared with pKSQ367, a reduction in the kinetics of TIL-killing of target A375 pKSQ398 cells was demonstrated. All of the groups have similar activities against the cell line, suggesting that the lower OKT3 affinity renders the target difficult for TIL to kill.

FIG. 15 shows that 10,000 A375-pKSQ367 or A375-pKSQ398 were plated in ultra-low-attachment plates and imaged over the course of 10 days to observe spheroid morphology.

FIG. 16 shows that 10,000 A375-pKSQ367 or A375-pKSQ398 were plated in ultra-low-attachment plates. Non-electroporated (noEP) and SOCS1-edited TIL were added after 4 days at various E:T ratios. Imaging was continuous throughout spheroid formation and coculture period. These images were collected after 36 hours for A375-pKSQ367 and 72 hours for A375-pKSQ398.

FIG. 17 shows that 10,000 A375-pKSQ367 or A375-pKSQ398 were plated in ultra-low-attachment plates. Non-electroporated (noEP) and SOCS1-edited TIL were added after 4 days at various E:T ratios. Imaging was continuous throughout spheroid formation and coculture period. These images were collected after 72 hours of coculture.

FIG. 18 shows that 10,000 A375-pKSQ367 were plated in ultra-low-attachment plates. Non-electroporated (noEP) and SOCS1-edited TIL were added after 4 days at various E:T ratios. Imaging was continuous throughout spheroid formation and coculture period. These images were collected after 5 days of coculture.

FIG. 19 shows that 5,000 or 10,000 A375-pKSQ398 were plated in ultra-low-attachment plates. Non-electroporated (noEP) and SOCS1-edited TIL were added after 4 days at various E:T ratios. Imaging was continuous throughout spheroid formation and coculture period. These images were collected after 5 days of coculture.

FIG. 20 shows cytotoxicity of SOCS1-edited TIL and control unedited TIL against A375-OKT3lt spheroids over 72 hours at different Effector:Target (E:T) ratios. In a monolayer culture, SOCS1-edited cells did not differ from unedited cells in the killing of both A375-pKSQA367 and A375-pKSQ368 cells (FIG. 13 and FIG. 14). In the 3D spheroid setting. SOCS1-edited cells showed enhanced cytotoxicity in comparison to control. This suggests that in a spheroid setting the assay has different sensitivity.

FIG. 21 shows IFNγ produced by SOCS1-edited TIL and control unedited TIL against A375-OKT3lt spheroids at 24 hours and different Effector:Target (E:T) ratios.

FIG. 22 shows IL-6 produced by SOCS1-edited TIL and control unedited TIL against A375-OKT3lt spheroids at 24 hours and different Effector:Target (E:T) ratios.

FIG. 23 shows luminescence level detected in supernatant after 24 hr TIL-spheroid co-culture.

FIG. 24 shows calculated LDH level in supernatant after 24 hr TIL-spheroid co-culture.

### DETAILED DESCRIPTION

The present disclosure provides, in some aspects, methods and compositions useful for measuring the antitumor potency of tumor infiltrating lymphocytes (TILs). In some embodiments, these methods include for example, coculturing TILs and immortalized cells, and assessing TIL potency using approaches that comprise coculturing TILs, immortalized cells, and bispecific molecules.

Many TIL potency assays currently being used for the monitoring of T cell function have focused on the use of cytokine (e.g., IFN-γ or IL-2) release assays. For example, T cell activation can be determined by measuring IFN-γ secretion following a short coculture period with non-cell-based T cell activation reagents, including anti-CD3/anti-CD28 antibody-coated beads. Cytokine secretion has been correlated with cytolytic activity of CD8⁺ T cells because cytokines, such as IFN-γ, enhance MHC I and Fas expression on target cells. For example, TILs may be considered potent if, for instance, interferon gamma (IFN-γ) release is greater than 50 μg/ml, greater than 100 μg/ml, greater than 150 μg/ml, or greater than 200 μg/ml upon TCR stimulation.

However, activation by and direct killing of target tumor cells, which requires that the TILs are capable at least of interacting with the target cells and producing/releasing mediators for death induction, such as degranulation of cytolytic granules containing granzyme B and perforin, is an equally important indicator of clinical efficacy that is not measured in presently available cytokine release assays, which are driven by non-cell-based TIL activation methods. The TIL potency assays provided herein improve upon the existing TIL potency assays (e.g., cytokine release assays) by activating TIL through relevant tumor cell-based interactions that afford the opportunity to directly measure cell death and/or viability of target cells (e.g., immortalized cells, such as cancer cells), or measure of surrogate markers of degranulation such as CD107a, or the production of effector cytokines/chemokines (e.g., proinflammatory cytokines/chemokines), such as IFN-γ, IL-6, IL_2, and TNFα, in a more physiologically relevant TIL/tumor-cell co-culture setting.

A “TIL potency assay” refers to an assay used to characterize, for example, quantify, antitumor activity (e.g., cytokine production, TIL degranulation, tumor growth inhibition) of TILs. TIL potency assays may be used for assessing TIL antitumor activity before and/or after rapid expansion of the TILs and prior to clinical use applications, such as adoptive cell therapy (ACT).

### Tumor Infiltrating Lymphocytes

Tumor infiltrating lymphocytes (TILs), including engineered TILs and/or edited TILs, may be characterized based on the potency of their antitumor activity (e.g., inhibition of tumor cell growth).

The phrase “tumor infiltrating lymphocytes” or “TILs” refers to a population of lymphocytes that have left the bloodstream of a subject and migrated into a tumor. TILs include, but are not limited to, CD8+ cytotoxic T cells, CD4+ T cells including Th1 and Th17 CD4+ T cells, natural killer T cells, and natural killer (NK) cells. TILs include both primary and secondary TILs. “Primary TILs” are those that are obtained from patient tissue samples as outlined herein (sometimes referred to as “freshly harvested”), and “secondary TILs” are any TIL cell populations that have been expanded or proliferated, including, but not limited to bulk TILs and expanded TILs (“REP TILs” or “post-REP TILs”). In some embodiments, primary TILs include tumor reactive T cells that are obtained from peripheral blood of a patient. TIL cell populations can include genetically modified or otherwise engineered TILs. “TILs” also refers to a population of lymphocytes that have left the blood stream of a subject, have migrated into a tumor and then have departed to again enter the bloodstream.

As generally outlined herein, TILs are generally taken from a patient sample and manipulated to expand their number prior to transplant into a patient. In some embodiments, the TILs may be genetically manipulated as discussed below. In general, TILs are initially obtained from a patient tumor sample (“primary TILs”) and then expanded into a larger population for further manipulation as described herein, optionally cryopreserved and restimulated, and optionally evaluated for phenotype and metabolic parameters as an indication of TIL health.

The terms “subject” and “patient” refer to a human being. In some embodiments, this human being may be a patient in need of immunotherapy involving an expanded population of the patient's own TILs. In other embodiments, this human being may be a patient in need of immunotherapy involving an expanded population of another patient's own TILs.

TILs can generally be defined either biochemically, using cell surface markers, or functionally, by their ability to infiltrate tumors and effect treatment. TILs can be generally categorized as expressing one or more of the following biomarkers: CD4, CD8, TCR αβ, TCRgd, CD27, CD28, CD56, CCR7, CD45RA, CD45RO, CD95, PD-1, and CD25. Additionally, and alternatively, TILs can be functionally defined by their ability to infiltrate solid tumors upon reintroduction into a patient.

Adoptive cell therapy utilizing TILs cultured ex vivo by conventional TIL manufacturing processes involves at least two steps, namely at least one rapid expansion protocol (REP) step subsequent to a pre-REP step. Adoptive cell therapy has resulted in successful therapy following host immunosuppression in patients with melanoma. Current infusion acceptance parameters rely on readouts of the composition of TILs (e.g., CD28, CD8, or CD4 positivity) and on the numerical folds of expansion and viability of the REP product.

The phrase “population of cells” or “population of TILs” refers to a number of cells or TILs that share common traits. In general, populations generally range from 1×10⁶ to 1×10¹⁰ in number, with different TIL populations comprising different numbers. For example, initial growth of primary TILs in the presence of IL-2 can result in a population of bulk TILs of roughly 1×10⁷ cells. REP expansion is generally done to provide populations of 1.5×10⁹ to 1.5×10¹⁰ cells for infusion. In some embodiments, the population of cells is monoclonal. In other embodiments, the population of cells is polyclonal. In some embodiments, when the population of cells is polyclonal, the cells still share one or more common traits. A monoclonal T cell population will result in the predominance of a single TCR-gene rearrangement pattern. In contrast, polyclonal T cell populations have diverse TCR-gene rearrangement pattern, which can make them more effective in certain situations.

In some embodiments, the TILs are genetically engineered to include additional functions including, but not limited to, a high-affinity T cell receptor (TCR), e.g., a TCR targeted at a tumor-associated antigen such as MAGE-1, HER2, or NY-ESO-1, or a chimeric antigen receptor (CAR) which binds to a tumor-associated cell surface molecule (e.g., mesothelin) or lineage-restricted cell surface molecule (e.g., EGFR, CD19 or HER2).

The term “engineered TIL” or “eTIL” encompasses TILs comprising one or more genomic modifications, effected through non-natural means, resulting in the reduced expression and/or function of one or more endogenous target genes as well as TILs comprising a non-naturally occurring gene-regulating system capable of reducing the expression and/or function of one or more endogenous target genes. An “unmodified TIL” or “control TIL” refers to a TIL or population of TILs wherein the genomes have not been modified through non-naturally occurring means and that does not comprise a non-naturally occurring gene-regulating system or comprises a control gene-regulating system (e.g., an empty vector control, a non-targeting gRNA, a scrambled siRNA, etc.). TILs that occur naturally that have reduced expression and/or function of one or more endogenous genes are included under the terms unmodified or control TILs.

In some embodiments, the engineered TILs manufactured by the methods described herein comprise one or more modifications (e.g., insertions, deletions, or mutations of one or more nucleic acids) in the genomic DNA sequence of an endogenous target gene resulting in the reduced expression and/or function the endogenous gene. In some embodiments, the modifications in the genomic DNA sequence reduce or inhibit mRNA transcription, thereby reducing the expression level of the encoded mRNA transcript and protein. In some embodiments, the modifications in the genomic DNA sequence reduce or inhibit mRNA translation, thereby reducing the expression level of the encoded protein. In some embodiments, the modifications in the genomic DNA sequence encode a modified endogenous protein with reduced or altered function compared to the unmodified (i.e., wild-type) version of the endogenous protein (e.g., a dominant-negative mutant, described infra).

In some embodiments, the modified TILs further comprise an engineered antigen-specific receptor recognizing a protein target expressed by a target cell, such as a tumor cell or an antigen presenting cell (APC). The term “engineered antigen receptor” refers to a non-naturally occurring antigen-specific receptor such as a chimeric antigen receptor (CAR) or a recombinant T cell receptor (TCR). In some embodiments, the engineered antigen receptor is a CAR comprising an extracellular antigen binding domain fused via hinge and transmembrane domains to a cytoplasmic domain comprising a signaling domain. In some embodiments, the CAR extracellular domain binds to an antigen expressed by a target cell in an MHC-independent manner leading to activation and proliferation of the RE cell. In some embodiments, the extracellular domain of a CAR recognizes a tag fused to an antibody or antigen binding fragment thereof. In such embodiments, the antigen-specificity of the CAR is dependent on the antigen-specificity of the labeled antibody, such that a single CAR construct can be used to target multiple different antigens by substituting one antibody for another. In some embodiments, the extracellular domain of a CAR may comprise an antigen binding fragment derived from an antibody. Antigen binding domains that are useful in the present disclosure include, for example, scFvs, antibodies, antigen binding regions of antibodies, variable regions of the heavy/light chains, and single chain antibodies.

In some embodiments, the intracellular signaling domain of a CAR may be derived from the TCR complex zeta chain (such as CD3ξ signaling domains), FcγRIII, FcεRI, or the Tlymphocyte activation domain. In some embodiments, the intracellular signaling domain of a CAR further comprises a costimulatory domain, for example a 4-1BB, CD28, CD40, MyD88, or CD70 domain. In some embodiments, the intracellular signaling domain of a CAR comprises two costimulatory domains, for example any two of 4-1BB, CD28, CD40, MyD88, or CD70 domains. Exemplary CAR structures and intracellular signaling domains are known in the art (See e.g., WO 2009/091826; US20130287748; WO 2015/142675; WO 2014/055657; and WO 2015/090229, incorporated herein by reference).

CARs specific for a variety of tumor antigens are known in the art, for example CD171-specific CARs (Park et al., Mol Ther (2007) 15(4):825-833), EGFRvIII-specific CARs (Morgan et al., Hum Gene Ther (2012) 23(10):1043-1053), EGF-R-specific CARs (Kobold et al., J Natl Cancer Inst (2014) 107(1):364), carbonic anhydrase K-specific CARs (Lamers et al., Biochem Soc Trans (2016) 44(3):951-959), FR-α-specific CARs (Kershaw et al., Clin Cancer Res (2006) 12(20):6106-6015), HER2-specific CARs (Ahmed et al., J Clin Oncol (2015) 33(15)1688-1696; Nakazawa et al., Mol Ther (2011) 19(12):2133-2143; Ahmed et al., Mol Ther (2009) 17(10):1779-1787; Luo et al., Cell Res (2016) 26(7):850-853; Morgan et al., Mol Ther (2010) 18(4):843-851; Grada et al., Mol Ther Nucleic Acids (2013) 9(2):32), CEA-specific CARs (Katz et al., Clin Cancer Res (2015) 21(14):3149-3159), IL13Rα2-specific CARs (Brown et al., Clin Cancer Res (2015) 21(18):4062-4072), GD2-specific CARs (Louis et al., Blood (2011) 118(23):6050-6056; Caruana et al., Nat Med (2015) 21(5):524-529), ErbB2-specific CARs (Wilkie et al., J Clin Immunol (2012) 32(5):1059-1070), VEGF-R-specific CARs (Chinnasamy et al., Cancer Res (2016) 22(2):436-447), FAP-specific CARs (Wang et al., Cancer Immunol Res (2014) 2(2):154-166), MSLN-specific CARs (Moon et al, Clin Cancer Res (2011) 17(14):4719-30), NKG2D-specific CARs (VanSeggelen et al., Mol Ther (2015) 23(10):1600-1610), CD19-specific CARs (Axicabtagene ciloleucel (Yescarta®) and Tisagenlecleucel (Kymriah®). See also, 82/337 Li et al., J Hematol and Oncol (2018) 11(22), reviewing clinical trials of tumor-specific CARs.

As generally outlined herein, TILs are generally taken from a patient sample and manipulated to expand their number prior to transplant into a patient. In some embodiments, the TILs may be genetically manipulated as discussed below. In general, TILs are initially obtained from a patient tumor sample (“primary TILs”) and then expanded into a larger population for further manipulation, optionally cryopreserved and re-stimulated, and optionally evaluated for phenotype and metabolic parameters as an indication of TIL health.

A patient tumor sample may be obtained using methods known in the art, generally via surgical resection, needle biopsy, or other means for obtaining a sample that contains a mixture of tumor and TIL cells. In general, the tumor sample may be from any solid tumor, including primary tumors, invasive tumors or metastases. The solid tumor may be of any cancer type, including, but not limited to, bladder cancer, brain cancer, breast cancer (including triple negative breast cancer), cervical cancer, colon and rectal cancer, stomach cancer, endometrial cancer, renal cancer, lip and oral cancer, head and neck cancer (including, for example, head and neck squamous cell carcinoma (HNSCC)) glioblastoma, glioblastoma multiforme, neuroblastoma, liver cancer, mesothelioma, lung cancer (including non-small cell lung cancer (NSCLC) and small cell lung cancer), skin cancer (including but not limited to squamous cell carcinoma, basal cell carcinoma, nonmelanoma skin cancer and melanoma), ovarian cancer, uveal cancer, uterine cancer, pancreatic cancer, prostate cancer, sarcoma, and thyroid cancer. In some embodiments, useful TILs are obtained from malignant melanoma tumors, as these have been reported to have particularly high levels of TILs. Primary lung. (including non-small cell lung cancer (NSCLC)), bladder, cervical and melanoma tumors or metastases thereof can be used to obtain TILs.

Once obtained, the tumor sample is generally fragmented using sharp dissection into small pieces of from about 1 to about 8 mm³, or from about 0.5 to about 4 mm³ with from about 2-3 mm³ being particularly useful. The TILs are cultured from these fragments using enzymatic tumor digests. Such tumor digests may be produced by incubation in enzymatic media (e.g., Roswell Park Memorial Institute (RPMI) 1640 buffer, 2 mM glutamate, 10 μg/ml gentamicin, 30 units/ml of DNase and 1.0 mg/ml of collagenase), followed by mechanical dissociation (e.g., using a tissue dissociator). Tumor digests may be produced by placing the tumor in enzymatic media and mechanically dissociating the tumor for approximately 1 minute, followed by incubation for 30 minutes at 37° C. in 5% CO₂, followed by repeated cycles of mechanical dissociation and incubation under the foregoing conditions until only small tissue pieces are present. At the end of this process, if the cell suspension contains a large number of red blood cells or dead cells, a density gradient separation using FICOLL branched hydrophilic polysaccharide may be performed to remove these cells. Alternative methods known in the art may be used, such as those described in U.S. Patent Application Publication No. 2012/0244133 A1, the disclosure of which is incorporated herein by reference in its entirety. Any of the foregoing methods may be used in any of the embodiments described herein for methods of expanding TILs or methods treating a cancer.

In general, the harvested cell suspension is called a “primary cell population” or a “freshly harvested” cell population. In some embodiments, fragmentation includes physical fragmentation, including for example, dissection as well as digestion. In some embodiments, the fragmentation is physical fragmentation. In some embodiments, the fragmentation is dissection. In some embodiments, the fragmentation is by digestion. In some embodiments, TILs can be initially cultured from enzymatic tumor digests and tumor fragments obtained from patients.

In some embodiments, the TILs are obtained from tumor digests. In some embodiments, tumor digests are generated by incubation of mechanically dissociated tumor in enzyme media, for example, but not limited to RPMI 1640, 2 mM GlutaMAX, 10 mg/ml gentamicin, 30 U/ml DNase, and 1.0 mg/ml collagenase, followed by mechanical dissociation (GentleMACS, Miltenyi Biotec, Auburn, Calif.). In some embodiments, the mechanically dissociated tumor would be broken up into approximately 1 mm³ pieces. After placing the tumor in enzyme media, the tumor can be mechanically dissociated for approximately 1 minute. The solution can then be incubated for 30 minutes at 37° C. in 5% CO₂ and can then be mechanically disrupted again for approximately 1 minute. After being incubated again for 30 minutes at 37° C. in 5% CO₂, the tumor can be mechanically disrupted a third time for approximately 1 minute. In some embodiments, after the third mechanical disruption if large pieces of tissue are present, one or two additional mechanical dissociations can be applied to the sample, with or without 30 additional minutes of incubation at 37° C. in 5% CO₂. In some embodiments, at the end of the final incubation if the cell suspension contains a large number of red blood cells or dead cells, a density gradient separation using FICOLL can be performed to remove these cells.

In some embodiments, cells can be optionally frozen or cryopreserved after sample harvest and stored frozen prior to entry into the expansion phase.

In some embodiments, the TILs are expanded for up to a total of 9, 10, 11, 12, 13, 14, 15, 16, 17, 18, 19, 20, 21, 22, 23, 24, 25, 26, 27 or 28 days from the initial tumor fragmentation or disaggregation. In some embodiments, the TILs are expanded for a total of 9-25 days, 9-21 days, or 9-14 days. In some embodiments, the TILs are expanded for up to a total of 9 days. In some embodiments, the TILs are expanded for up to a total of 10 days. In some embodiments, the TILs are expanded for up to a total of 11 days. In some embodiments, the TILs are expanded for up to a total of 12 days. In some embodiments, the TILs are expanded for up to a total of 13 days. In some embodiments, the TILs are expanded for up to a total of 14 days. In some embodiments, the TILs are expanded for up to a total of 15 days. In some embodiments, the TILs are expanded for up to a total of 16 days. In some embodiments, the TILs are expanded for up to a total of 17 days. In some embodiments, the TILs are expanded for up to a total of 18 days. In some embodiments, the TILs are expanded for up to a total of 19 days. In some embodiments, the TILs are expanded for up to a total of 20 days. In some embodiments, the TILs are expanded for up to a total of 21 days. In some embodiments, the TILs are expanded for up to a total of 22 days. In some embodiments, the TILs are expanded for up to a total of 23 days. In some embodiments, the TILs are expanded for up to a total of 24 days. In some embodiments, the TILs are expanded for up to a total of 25 days. In some embodiments, the TILs are expanded for up to a total of 26 days. In some embodiments, the TILs are expanded for up to a total of 27 days. In some embodiments, the TILs are expanded for up to a total of 28 days.

In some embodiments, the expanded TILs are analyzed for expression of numerous phenotype markers, including those described herein. In some embodiments, the marker is selected from: TCRα/β, CD57, CD28, CD4, CD27, CD56, CD8a, CD45RA, CD45RO, CD8a, CCR7, CD4, CD3, CD38, and HLA-DR. In some embodiments, expression of one or more regulatory markers is measured, namely from the group: CD137, CD8a, Lag3, CD4, CD3, PD-1. TIM-3, CD69, CD8a, TIGIT, CD4, CD3, KLRG1, and CD154.

In some embodiments, the memory marker is CCR7 or CD62L. In embodiments, restimulated TILs are evaluated for cytokine release, using cytokine release assays. In some embodiments, TILs are evaluated for interferon-gamma (IFN-γ) secretion in response to stimulation either with OKT3 or coculture with autologous tumor digest. In some embodiments, TILs are evaluated for IL-6 secretion in response to stimulation either with OKT3 or coculture with autologous tumor digest. Additional effector cytokines that could be measured include, but are not limited to, IL-1, IL-2, IL-12, IL-17, IL-18, granulocyte-macrophage colony stimulating factor (GM-CSF), and tumor necrosis factor-α (TNFα). Chemokines such as CXCL10, CXCL13, CCL1, CCL3, CCL4, CCL5, CCL9/10, CCL17, CCL22, CCL23, and XCL1 can also be evaluated.

TILs are evaluated for various regulatory markers, such as TCRα/β, CD56, CD27, CD28, CD57, CD45RA, CD45RO, CD25, CD127, CD95, IL-2R, CCR7, CD62L, KLRG1, and CD122

### Immortalized Cells

Immortalized cells are cells that have been manipulated to proliferate indefinitely and can thus be cultured for long periods of time. Immortalized cell lines are typically derived from a variety of sources that have chromosomal abnormalities or mutations that permit them to continually divide, such as tumors. Immortalized cells are thus considered to be “engineered.” A population of immortalized cells may be a heterogenous population or may be derived from a single immortalized clone (to form a clonal population). In some embodiments, immortalized cells comprise a heterogenous population of immortalized cells. In some embodiments, immortalized cells comprise a clonal population of immortalized cells.

Immortalized cells can be derived from a variety of species and/or origins. For example, immortalized cells can be immortalized animal cells or immortalized human cells, or a combination thereof. In some embodiments, immortalized cells are immortalized human cells.

In some embodiments, immortalized cells are cancer cells. For example, cancer cells may include, but are not limited to, melanoma cells, colorectal cancer cells, bile duct cancer cells, and breast cancer cells. In some embodiments, the cancer cells are selected from melanoma cells, colorectal cancer cells, bile duct cancer cells, and breast cancer cells.

Immortalized cells that may be engineered for use in the TIL potency assays described herein may include, but are not limited to, A375 melanoma cells (e.g., ATCC®) CRL-1619™), K562 multipotential, hematopoietic malignant cells, primary cells (e.g., ATCC® CCL-243™), human embryonic kidney (HEK) 293T cells (e.g., ATCC® CRL-1573), and Chinese hamster ovary (CHO) cells (e.g., ATCC® CCL-61™). In some embodiments, the immortalized cells are selected from A375 cells, K562 cells, primary cells, HEK293T cells, and CHO cells. It should be understood that the immortalized cells useful for the assays and methods described herein are not limited to the foregoing examples. Other immortalized cell lines are known in the field and may be used in accordance with the present disclosure.

The use of a cell line expressing immune suppressive markers to suppress T cell responses, such as PD-L1, PD-L2, to test whether TILs are resistant to suppressive signals are also contemplated herein. In some embodiments, immortalized cells that may be engineered for use in TIL potency assays comprise a cell line expressing immune suppressive markers to suppress T cell responses, such as PD-L1.

Cell lines that express co-stimulatory markers to enhance T cell responses, such as CD80/86, OX40L, and/or 41BBL may also be engineered for use in a TIL potency assay. In some embodiments, immortalized cells that may be engineered for in TIL potency assays comprise a cell line may express co-stimulatory markers to enhance T cell responses, such as CD80/86.

The phrase “tumor cells” or “cancer cells” refers to cells that divide in an uncontrolled manner, forming solid tumors or flooding the blood with abnormal cells. Healthy cells stop dividing when there is no longer a need for more daughter cells, but tumor cells or cancer cells continue to produce copies. They are also able to spread from one part of the body to another in a process known as metastasis. Tumor cells can be isolated from a number of cancer types including bladder cancer, brain cancer, breast cancer (including triple negative breast cancer), cervical cancer, colon and rectal cancer, stomach cancer, endometrial cancer, renal cancer, lip and oral cancer, head and neck cancer (including, for example, head and neck squamous cell carcinoma (HNSCC)) glioblastoma, glioblastoma multiforme, neuroblastoma, liver cancer, mesothelioma, lung cancer (including non-small cell lung cancer (NSCLC) and small cell lung cancer), skin cancer (including but not limited to squamous cell carcinoma, basal cell carcinoma, nonmelanoma skin cancer and melanoma), ovarian cancer, uveal cancer, uterine cancer, pancreatic cancer, prostate cancer, sarcoma, and thyroid cancer. In some embodiments, cancer cells are also isolated from lymphoma. Tumor cells can be isolated from primary tumors and metastases.

While immortalized cells are described throughout in the connect of coculturing with TILs, it should be understood that the immortalized cells may be replaced with any tumor cells, such as cancer cells, that express or have been engineered to express a molecule that activates a T cell, e.g., binds to a T cell antigen.

Molecules that Activate Lymphocytes

Engineered immortalized cells of the present disclosure may comprise or express a molecule that activates a T cell.

A “molecule that activates a lymphocyte” and a “molecule that activates a T cell” refers to a nonendogenous stimulus that causes the cell to become activated. In the endogenous process, T cells, for example, become activated when they are presented with peptide antigens by MHC class II molecules, which are expressed on the surface of antigen-presenting cells (APCs). Once activated, the T cells divide rapidly and secrete cytokines that regulate or assist the immune response. The endogenous T cell activation process involves at least (a) activation of the TCR complex, which involves CD3, and (b) co-stimulation of CD28 or 4-1BB by proteins on the APC surface. It is known in the art that the endogenous activation of T cells can be simulated by stimulation of T cells by CD3, CD28 or 4-1BB agonists (e.g., antibodies). Thus, CD3, CD28 and/or 4-1BB can together activate T cells.

Activated T cells increase in number or proliferate and begin producing cytokines (activated TILs) to boost the immune response.

Immortalized cells may comprise and/or express a molecule that activates a T cell by binding (e.g., directly binding) to a T cell antigen. In some embodiments, TILs (e.g., engineered TILs and/or edited TILs) express a T cell antigen. Non-limiting examples of T cell antigens include CD3, CD28, CD2, 41BB, OX40, GITR, ICOS, CD4, CD8. In some embodiments, the T cell antigen is CD3. In some embodiments, the T cell antigen is CD28. In some embodiments, the T cell antigen is CD2.

The term “CD3” refers to the CD3 (cluster of differentiation 3) T cell co-receptor that helps to activate both the cytotoxic T cell (CD8+ naïve T cells) and also T helper cells (CD4+ naïve T cells). CD3 is a protein complex composed of six distinct polypeptide chains (2 CD3 zeta chains, 2 CD3 epsilon chains, 1 CD3e gamma chain, and 1 CD3 delta chain). These chains associate with the T cell receptor (TCR) alpha and beta chains (or gamma and delta chains) to generate an activation signal in T lymphocytes. The TCR alpha and beta chains (or gamma and delta chains), and CD3 molecules together constitute the TCR complex. The human CD3E gene is identified by National Center for Biotechnology Information (NCBI) Gene ID 916. An exemplary nucleotide sequence for a human CD3E gene is the NCBI Reference Sequence: NG_007383.1.

The term “CD28” refers to cluster of differentiation 28, which is one of the proteins expressed on T cells that provides co-stimulatory signals required for T cell activation and survival. T cell stimulation through CD28 in addition to the T cell receptor (TCR) can provide a potent signal for the production of various cytokines, such as interleukins. CD28 is the receptor for CD80 and CD86 proteins. When activated by Toll-like receptor ligands, CD80 expression is upregulated in antigen-presenting cells (APCs). The human CD28 gene is identified by NCBI Gene ID 940. An exemplary nucleotide sequence for a human CD28 gene is the NCBI Reference Sequence: NG_029618.1. An exemplary amino acid sequence of a human CD28 polypeptide is:

(SEQ ID NO: 19)
MLRLLLALNLFPSIQVTGNKILVKQSPMLVAYDNAVNLSCKYSYNLFSRE
FRASLHKGLDSAVEVCVVYGNYSQQLQVYSKTGFNCDGKLGNESVTFYLQ
NLYVNQTDIYFCKIEVMYPPPYLDNEKSNGTIIHVKGKHLCPSPLFPGPS
KPFWVLVVVGGVLACYSLLVTVAFIIFWVRSKRSRLLHSDYMNMTPRRPG
PTRKHYQPYAPPRDFAAYRS

The term “CD2” refers to cluster of differentiation 2, which is a cell adhesion molecule found on the surface of T cells and natural killer (NK) cells. CD2 interacts with other adhesion molecules and acts as a co-stimulatory molecule on T and NK cells. The human CD2 gene is identified by NCBI Gene ID 914. An exemplary nucleotide sequence for a human CD2 gene is the NCBI Reference Sequence: NG_050908.1. An exemplary amino acid sequence of a human CD2 polypeptide is:

(SEQ ID NO: 20)
MSFPCKFVASFLLIFNVSSKGAVSKEITNALETWGALGODINLDIPSFQM
SDDIDDIKWEKTSDKKKIAQFRKEKETFKEKDTYKLFKNGTLKIKHLKTD
DQDIYKVSIYDTKGKNVLEKIFDLKIQERVSKPKISWTCINTTLTCEVMN
GTDPELNLYQDGKHLKLSQRVITHKWTTSLSAKFKCTAGNKVSKESSVEP
VSCPEKGLDIYLIIGICGGGSLLMVFVALLVFYITKRKKQRSRRNDEELE
TRAHRVATEERGRKPHQIPASTPQNPATSQHPPPPPGHRSQAPSHRPPPP
GHRVQHQPQKRPPAPSGTQVHQQKGPPLPRPRVQPKPPHGAAENSLSPSS
N.

In some embodiments, immortalized cells comprise a molecule that activates a T cell by binding, e.g., specifically binding, to a T cell antigen. In some embodiments, immortalized cells comprise a molecule that activates a T cell by binding to a CD3 antigen. In some embodiments, immortalized cells comprise a molecule that activates a T cell by binding to a CD28 antigen. In some embodiments, immortalized cells comprise a molecule that activates a T cell by binding to a CD2 antigen.

The phrase “specifically binding” refers to a molecule (e.g., antibody) interacting with high specificity with a particular antigen (e.g., T cell antigen), as compared with other antigens for which the complex has a lower affinity to associate. The specific binding interaction can be mediated through ionic bonds, hydrogen bonds, or other types of chemical or physical associations. In some embodiments, the molecule specifically binds a particular antigen when it recognizes its target antigen in a complex mixture of proteins and/or macromolecules. In some embodiments, the molecule that activates a T cell binds to a T cell antigen with an affinity (KD) of approximately less than 10⁻⁵ M, such as approximately less than 10⁻⁶ M, 10⁻⁷ M, 10⁻⁸ M, 10⁻⁹ M or 10⁻¹⁰ M or even lower.

In some embodiments, a molecule that activates a T cell is a T cell agonist. The term “agonist” refers to a chemical, a molecule, a macromolecule, a complex of molecules, or a complex of macromolecules that binds to a target, either on the surface of a cell or in soluble form. In certain embodiments, when an agonist binds to a target on the surface of a cell, the agonist activates the target to produce a biological response. Agonists include hormones, neurotransmitters, antibodies, and fragments of antibodies.

Non-limiting examples of molecules that activate T cells include, but are not limited to, antibodies, such as whole antibodies and/or antibody fragments, NANOBODY® binders, AFFIMER® binders, and other molecular binders, such as ligands and receptors.

The term “antibody” refers to an immunoglobulin (Ig) molecule, which is generally comprised of four polypeptide chains, two heavy (H) chains and two light (L) chains, or a functional fragment, mutant, variant, or derivative thereof, that retains the epitope binding features of an Ig molecule. Such fragment, mutant, variant, or derivative antibody formats are known in the art. In an embodiment of a full-length antibody, each heavy chain is comprised of a heavy chain variable region (VH) and a heavy chain constant region (CH). The heavy chain variable region (domain) is also designated as VDH in this disclosure. The CH is comprised of three domains, CH1, CH2 and CH3. Each light chain is comprised of a light chain variable region (VL) and a light chain constant region (CL). The CL is comprised of a single CL domain. The light chain variable region (domain) is also designated as VDL in this disclosure. The VH and VL can be further subdivided into regions of hypervariability, termed complementarity determining regions (CDRs), interspersed with regions that are more conserved, termed framework regions (FRs). Generally, each VH and VL is composed of three CDRs and four FRs, arranged from amino-terminus to carboxy-terminus in the following order: FR1, CDR1, FR2, CDR2, FR3, CDR3, and FR4. Immunoglobulin molecules can be of any type (e.g., IgG, IgE, IgM, IgD, IgA and IgY), class (e.g., IgG1, IgG2, IgG3, IgG4, IgA1 and IgA2), or subclass.

In some embodiments, immortalized cells express a molecule that is an antibody fragment. In some embodiments, an antibody fragment is selected from a single-chain variable fragment (scFv), a F(ab′)2 fragment, a Fab fragment, a Fab′ fragment, and an Fv fragment.

The term “fragment” used in association with agonist or antibody, refers to a fragment of the agonist or antibody that retains the ability to specifically bind to an antigen. Examples of fragments of antibodies include (i) an Fab fragment, a monovalent fragment consisting of the VL, VH, CL and CH1 domains; (ii) an F(ab′)2 fragment, a bivalent fragment comprising two Fab fragments linked by a disulfide bridge at the hinge region; (iii) an Fd fragment consisting of the VH and CH1 domains; (iv) an Fv fragment consisting of the VL and VH domains of a single arm of an antibody; (v) a dAb fragment, which comprises a single variable domain; and (vi) an isolated complementarity determining region (CDR). Furthermore, although the two domains of the Fv fragment, VL and VH, are encoded by separate genes, they can be joined, using recombinant methods, by a synthetic linker that enables them to be made as a single protein chain in which the VL and VH regions pair to form monovalent molecules (known as single chain Fv (scFv)). Such single chain antibodies are also intended to be encompassed within the term “antigen-binding portion” of an antibody. Other forms of single chain antibodies, such as diabodies are also encompassed. In addition, single chain antibodies also include “linear antibodies” comprising a pair of tandem Fv segments (VH-CH1-VH-CH1), which, together with complementary light chain polypeptides, form a pair of antigen binding regions.

The term “KD” refers to the dissociation equilibrium constant of a particular agonist-antigen interaction. Typically, the agonists described herein bind to a target with a dissociation equilibrium constant (KD) that is higher than the KD of mOKT3 scFv, which is about 1×10⁻⁹ M or 1×10⁻¹⁰ M, for example, as determined using surface plasmon resonance (SPR) technology in a Biacore instrument using the agonist as the ligand and the target as the analyte. In some embodiments, the agonists described herein (e.g., a low-affinity mOKT3 scFv variant) bind to a target protein (e.g., CD3) with an affinity corresponding to a KD that is higher than 5×10⁻¹⁰ M.

The term “koff” (sec−1) refers to the dissociation rate constant of a particular agonist-antigen interaction. Said value is also referred to as the kd value.

The term “kon” (M−1×sec−1) refers to the association rate constant of a particular agonist-antigen interaction.

The term “KD” (M) refers to the dissociation equilibrium constant of a particular agonist-antigen interaction

The term “KA” (M⁻¹) refers to the association equilibrium constant of a particular agonist-antigen interaction and is obtained by dividing the kon by the koff.

The phrase “anti-CD28 antibody” refers to an antibody or variant thereof, e.g., a monoclonal antibody, and includes human, humanized, chimeric or murine antibodies which are directed against the CD28 receptor in the T cell antigen receptor of mature T cells.

The phrase “anti-CD2 antibody” refers to an antibody or variant thereof, e.g., a monoclonal antibody, and includes human, humanized, chimeric or murine antibodies which are directed against the CD2 receptor in the T cell antigen receptor of mature T cells.

The phrase “anti-CD3 antibody” refers to an antibody or variant thereof, e.g., a monoclonal antibody, and includes human, humanized, chimeric or murine antibodies which are directed against the CD3 receptor in the T cell antigen receptor of mature T cells (see, e.g., International Publication No. WO2013186613A1, incorporated herein by reference). Anti-CD3 antibodies include OKT3, also known as muromonab. Anti-CD3 antibodies also include the UCHT1 clone, also known as T3 and CD3c. Other anti-CD3 antibodies include, for example, otelixizumab, teplizumab, and visilizumab.

Muromonab-CD3 light chain
(SEQ ID NO: 17)
QIVLTQSPAIMSASPGEKVTMTCSASSSVSYMNWYQQKSGTSPKRWIYDT
SKLASGVPAHFRGSGSGTSYSLTISGMEAEDAATYYCQQWSSNPFTFGSG
TKLEINRADTAPTVSIFPPSSEQLTSGGASVVCFLNNFYPKDINVKWKID
GSERQNGVLNSWTDQDSKDSTYSMSSTLTLTKDEYERHNSYTCEATHKTS
TSPIVKSFNRNEC
Muromonab-CD3 heavy chain
(SEQ ID NO: 18)
QVQLQQSGAELARPGASVKMSCKASGYTFTRYTMHWVKQRPGQGLEWIGY
INPSRGYTNYNQKFKDKATLTTDKSSSTAYMQLSSLTSEDSAVYYCARYY
DDHYCLDYWGOGTTLTVSSAKTTAPSVYPLAPVCGGTTGSSVTLGCLVKG
YFPEPVTLTWNSGSLSSGVHTFPAVLQSDLYTLSSSVTVTSSTWPSQSIT
CNVAHPASSTKVDKKIEPRPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK

The term “OKT3” refers to the anti-CD3 antibody produced by Miltenyi Biotech, Inc., San Diego, Calif., USA) and or biosimilar or variant thereof (e.g., a humanized, chimeric, or affinity matured variant). A hybridoma capable of producing OKT3 is available in the American Type Culture Collection and assigned the ATCC accession number CRL 8001. A hybridoma capable of producing OKT3 is available in the European Collection of Authenticated Cell Cultures (ECACC) and assigned Catalogue No. 86022706.

In some embodiments, the antibody fragment is an OKT3 antibody, such as an OKT3 antibody fragment. In some embodiments, an OKT3 antibody fragment is a membrane-bound OKT3 (mOKT3) scFv fragment.

Transmembrane domains may be utilized to anchor mOKT3 scFv to the cell surface of immortalized cells. For example, the human CD8 transmembrane domain can be utilized to anchor mOKT3 scFv to the cell surface of immortalized cells. The use of CD8 transmembrane domains from other species, such as mouse (pKSQ366), or other transmembrane proteins, such as CD14 or CD28 to anchor mOKT3 scFv to the cell surface of immortalized cells is also contemplated herein. In some embodiments, mOKT3 scFv is expressed tethered to the cell surface of the immortalized cells. In some embodiments, human CD8 transmembrane domain is utilized to anchor mOKT3 scFv to the cell surface of immortalized cells.

Many T cells will respond to the very strong stimulation of mOKT3. The use of lower affinity T cell receptor binding may allow for the separation of subtle differences in T cell receptor signaling thresholds due to cell-to-cell variation and may allow more sensitive quality determinations between T cell therapy products prior to infusion into patients. Low-affinity variants of mOKT3 fragments can be made via site mutagenesis to model more closely the affinities of natural T cell receptors with their recognized antigens. Published affinities of natural T cell receptors are in the μM KD range.

In some embodiments, a low-affinity mOKT3 scFv variant is used. In some embodiments, a molecule binds to CD3 with a dissociation constant (KD) that is lower than the KD of mOKT3 scFv, wherein the KD of mOKT scFv is about 1×10⁻⁹ to about 1×10⁻¹¹, for example, about 5×10⁻¹⁰ M. In some embodiments, the low-affinity mOKT3 scFv variant comprises an amino acid sequence having a R55 mutation, relative to the amino acid sequence of SEQ ID NO: 2. In some embodiments, the low-affinity mOKT3 scFv variant comprises an amino acid sequence having a Y57 mutation, relative to the amino acid sequence of SEQ ID NO: 2. In some embodiments, the low-affinity mOKT3 scFv variant comprises an amino acid sequence having R55 and Y57 mutations, relative to the amino acid sequence of SEQ ID NO: 2. In some embodiments, the low-affinity mOKT3 scFv variant comprises an amino acid sequence having R55M and Y57A mutations, relative to the amino acid sequence of SEQ ID NO: 2. In some embodiments, the low-affinity mOKT3 scFv variant comprises an amino acid sequence having R55L and Y57T mutations, relative to the amino acid sequence of SEQ ID NO: 2.

In some embodiments, the low-affinity mOKT3 scFv variant binds to CD3 with a dissociation constant (KD) of about 1×10⁻⁶ to about 5×10⁻⁸. For example, the low-affinity mOKT3 scFv variant may bind to CD3 with a KD of about 1×10⁻⁶, 1×10⁻⁷, or 1×10⁻⁸. In some embodiments, the low-affinity mOKT3 scFv variant binds to CD3 with a KD of about 5×10⁻⁷.

In some embodiments, the low-affinity mOKT3 scFv variant binds to CD3 with a KD that is least 10-fold lower, at least 20-fold lower, at least 30-fold lower, at least 40-fold lower, at least 50-fold lower, at least 60-fold lower, at least 70-fold lower, at least 80-fold lower, at least 90-fold lower, at least 100-fold lower, at least 110-fold lower, at least 120-fold lower, at least 130-fold lower, at least 140-fold lower, at least 150-fold lower, at least 160-fold lower, at least 170-fold lower, at least 180-fold lower, at least 190-fold lower, at least 200-fold lower, at least 210-fold lower, at least 220-fold lower, at least 230-fold lower, at least 240-fold lower, at least 250-fold lower, at least 275-fold lower, at least 300-fold, at least 400-fold, at least 500-fold, at least 600-fold, at least 700-fold, at least 800-fold, at least 900-fold, at least 1000-fold, at least 1100-fold, at least 1200-fold lower than the KD of mOKT3 scFv. In some embodiments, the low-affinity mOKT3 scFv variant binds to CD3 with a dissociation constant KD that is least 250-fold lower than the KD of mOKT3 scFv. In some embodiments, the low-affinity mOKT3 scFv variant binds to CD3 with a dissociation constant KD that is least 1000-fold lower than the KD of OKT3 scFv.

In some embodiments, a molecule that activates a T cell is a membrane-tethered molecule. Other molecules, such as bacterial superantigens (e.g., SEB), phytohaemagglutinin (PHA) or concanavalin A (ConA), that bind and cluster the T cell receptor CD3 to activate the T cell are contemplated to work in a similar manner to the mOKT3 scFv tethered to the cell surface of immortalized cells. In some embodiments, a molecule that activates a T cell is phytohaemagglutinin (PHA). In some embodiments, a molecule that activates a T cell is concanavalin A (ConA). Other monoclonal antibody scFv fragments that bind a T cell receptor/CD3 component, such as anti-CD3 antibody clone BC3, are also contemplated to work in a similar manner to the mOKT3 scFv tethered to the cell surface of immortalized cells. In some embodiments, a molecule that activates a T cell is an anti-CD3 antibody clone BC3.

In some embodiments, the immortalized cells express an Fc receptor. In such embodiments, an antibody, such as a monoclonal anti-CD3 antibody, that binds both the Fc receptor and a T cell antigen, such as CD3, may be used to assess the potency of the TILs.

In other embodiments, the molecule that activates a T cell is not expressed by the immortalized cell. Rather, the molecule may be a bispecific molecule that can bind to both the immortalized cell and to the T cell. Bispecific T cell engagers, such as blinatumomab, is one non-limiting example of a such a molecule that binds to CD19 expressed by the immortalized cells and CD3 expressed by T cells.

### Coculture Conditions

The methods provided herein, in some aspects, comprise coculturing lymphocytes (e.g., TILs) and immortalized cells.

The immortalized cells may be cultured as a two-dimensional monolayer of cells or as multilayer, three-dimensional spheroids. Spheroids are self-assembling multicellular aggregates that form in an environment that prevents attachment to a flat surface.

A two-dimensional culture is an adherent culture in which cells grow as a monolayer, for example, in a culture flask, dish, or multiwell plate having an adherent surface. By comparison, a three-dimensional spheroid culture is a nonadherent or low-adherent culture system in which cells grow as multilayer spheroids, for example, in suspension culture on non-adherent plates, in concentrated medium or in a gel-like substance, or on a scaffold.

In some embodiments, the immortalized cells of a three-dimensional system are cultured on an ultra-low attachment (ULA) surface. An ultra-low attachment surface is a surface that includes a substance that inhibits specific and nonspecific immobilization, forcing cells into a suspended state, enabling three-dimensional spheroid formation. In some embodiments, an ultra-low attachment surface comprises a hydrophilic, neutrally charged coating. In some embodiments, the hydrophilic, neutrally charged hydrogel coating is covalently bound to the surface. In some embodiments, the surface comprises polystyrene. Thus, in some embodiments, an ultra-low attachment surface is a polystyrene surface to which a hydrophilic, neutrally charged coating is covalently bound. As is known in the art, ultra-low attachment surfaces are generally stable, noncytotoxic, biologically inert, and non-degradable.

In some embodiments, the immortalized cells of a three-dimensional system are cultured in an ultra-low attachment multiwell plate (e.g., Corning®). In some embodiments, the ultra-low attachment surface of the multiwell plate (e.g., multiwell polystyrene plate) is a covalently bound hydrogel layer that is hydrophilic and neutrally charged. Various multiwell formats are available, for example, 6-, 24-, or 96-well formats may be used.

The culture conditions provided below may be used for either two-dimensional or three-dimensional cocultures.

In some embodiments, a coculture of immortalized cells includes 25,000 to 200,000 immortalized cells. For example, a coculture of immortalized cells may include 50,000 to 100,000 immortalized cells. In some embodiments, a coculture of immortalized cells includes 25000, 30000, 40000, 50000, 60000, 70000, 80000, 90000, or 10000 immortalized cells.

In some embodiments, the immortalized cells are plated, for example, in a multiwell plate, such as a 96-well plate, in media (e.g., Dulbecco's Modified Eagle Medium (DMEM)) with serum (e.g., fetal bovine serum (FBS)) and an antibiotic (e.g., Pen/Strep).

In some embodiments, the immortalized cells are cultured (e.g., in a multiwell plates with a ULA surface or in adherent multiwell plates) in media for about 48 hours to about 96 hours (e.g., about 48 hours, about 60 hours, about 72 hours, about 84 hours, or about 96 hours) to form three-dimensional spheroids prior to the addition of TILs.

To generate TILs, for example, such as pre-REP TILs, prior to coculture, in some embodiments, tumor digest samples (e.g., melanoma tumor digest samples) are thawed and plated (e.g., 1.5e6 cells/mL) in pre-REP media in the presence of IL-2. Re-plating and feeding of IL-2, in some embodiments, is repeated every 1-3 days until growth slows to less than 1.5× growth after two days, for example.

The day before coculture initiation, in some embodiments, pre-REP TILs are thawed and rested overnight in REP media (e.g., RPMI, AIM-V, 5% Human AB Serum and IL-2).

In some embodiments, pan T cells are isolated from peripheral blood mononuclear cells (PBMCs) and cocultured with the immortalized cells.

In some embodiments, cocultures are performed at a 2:1 effector (T cell) to target (immortalized cell) ratio (abbreviated as E:T). In some embodiments, cocultures are performed at a 1:1 E:T ratio. In some embodiments, cocultures are performed at a 1:2 E:T ratio. In some embodiments, cocultures are performed at a 3:1, 2:1, or 1:1 E:T ratio. In some embodiments, cocultures are performed at a 5:1 E:T ratio. In some embodiments, cocultures are performed at a 10:1 E:T ratio. In some embodiments, cocultures are performed at a 20:1 E:T ratio.

The period of coculture may vary. In some embodiments, TILs and immortalized cells are cocultured for at least 2, at least 3, or at least 4 hours. For example, TILs and immortalized cells may be cocultured for 2 to 72 hours, 2 to 48 hours, 2 to 36 hours, 2 to 24 hours, 2 to 12 hours, 4 to 72 hours, 4 to 48 hours, 4 to 36 hours, 4 to 24 hours, 4 to 12 hours, 12 to 72 hours, 8 to 72 hours, 8 to 48 hours, 8 to 36 hours, 8 to 24 hours, 8 to 12 hours, 12 to 72 hours, 12 to 48 hours, 12 to 36 hours, 12 to 24 hours, 24 to 72 hours, 24 to 48 hours, or 24 to 36 hours. In some embodiments, TILs and immortalized cells are cocultured for about 2 hours, 4 hours, 6 hours, 8 hours, 12 hours, 18 hours, 24 hours, 36 hours, 48 hours, or 72 hours. In some embodiments, TILs and immortalized cells are cocultured for at least 12 hours, at least 24 hours, at least 48 hours, at least 36 hours, or for at least 72 hours.

In some embodiments, a coculture of TILs and immortalized cells includes interleukin-2 (IL-2), IL-15, or a combination thereof, for example, at a final concentration of about 5,000 U/ml to about 8,000 U/ml (e.g., 5,000 U/ml, 5,500 U/ml, 6,000 U/ml, 6,500 U/ml, 7,000 U/ml, 7,500 U/ml, or 8,000 U/ml).

IL-2 is an interleukin, a type of cytokine signaling molecule in the immune system. It is a 15.5-16 kDa protein that regulates the activities of white blood cells (leukocytes, often lymphocytes) that are responsible for immunity. IL-2 is part of the body's natural response to microbial infection. IL-2 mediates its effects by binding to IL-2 receptors, which are expressed by lymphocytes. The major sources of IL-2 are activated CD4+ T cells and activated CD8+ T cells.

IL-2 has essential roles in key functions of the immune system, tolerance and immunity, primarily via its direct effects on T cells. In the thymus, where T cells mature, it prevents autoimmune diseases by promoting the differentiation of certain immature T cells into regulatory T cells, which suppress other T cells that are otherwise primed to attack normal healthy cells in the body. IL-2 enhances activation-induced cell death (AICD). IL-2 also promotes the differentiation of T cells into effector T cells and into memory T cells when the initial T cell is also stimulated by an antigen, thus helping the body fight off infections. Together with other polarizing cytokines, IL-2 stimulates naive CD4+ T cell differentiation into Th1 and Th2 lymphocytes while it impedes differentiation into Th17 and follicular Th lymphocytes. Its expression and secretion are tightly regulated and functions as part of both transient positive and negative feedback loops in mounting and dampening immune responses. Through its role in the development of T cell immunologic memory, which depends upon the expansion of the number and function of antigen-selected T cell clones, it plays a role in enduring cell-mediated immunity.

IL-15 is 14-15 kDa glycoprotein encoded by the 34 kb region of chromosome 4q31 in humans. IL-15 is a cytokine with structural similarity to IL-2. Like IL-2, IL-15 binds to and signals through a complex composed of IL-2/IL-15 receptor beta chain (CD122) and the common gamma chain (gamma-C, CD132). IL-15 is constitutively expressed by a large number of cell types and tissues, including monocytes, macrophages, dendritic cells (DC), keratinocytes, fibroblasts, myocyte and nerve cells. As a pleiotropic cytokine, it plays an important role in innate and adaptive immunity. IL-15 regulates the activation and proliferation of T and natural killer (NK) cells. Survival signals that maintain memory T cells in the absence of antigen are provided by IL-15. This cytokine is also implicated in NK cell development.

### Assessment of Lymphocyte Antitumor Potency

Following coculturing of lymphocytes (e.g., TILs) and immortalized cells, lymphocyte antitumor potency may be assessed, for example, by measuring degranulation (e.g., CD107a expression), cytokine production (e.g., IFN-γ) and/or cell death and/or viability of immortalized cells over a period of time. For example, a period of time for assessing potency may be 12 to 72 hours. In some embodiments, lymphocyte (e.g., TIL) antitumor potency is assessed for 12 to 48 hours, 12 to 36 hours, 12 to 24 hours, 24 to 72 hours, 24 to 48 hours, or 24 to 36 hours. In some embodiments, lymphocyte (e.g., TIL) antitumor potency is assessed for 12 hours, 24 hours, 36 hours, 48 hours, or 72 hours. In some embodiments, lymphocyte (e.g., TIL) antitumor potency is assessed for at least 12 hours, at least 24 hours, at least 48 hours, at least 36 hours, or for at least 72 hours.

Methods for measuring degranulation include, but are not limited to, cell surface staining of lysosomal-associated membrane glycoproteins (LAMPs), such as CD107a or CD107b, using flow cytometry readout. LAMPs are found on the lipid bilayer of cytolytic granules, which, upon release for mediation of killing by TIL, are fused to the TIL's surface, serving as a marker for degranulation and direct cytotoxicity.

Methods for measuring cytokine product include, but are not limited to, ELISA, Luminex or MSD assay of cell culture supernatants following co-culture of the mOKT3-A375 cell line with TIL, or qRT-PCR analysis of cytokine transcripts in TIL following mOTK3-A375 co-culture.

Methods for measuring cell death and/or viability to assess potency include, but are not limited to, real-time cell viability assays, ATP cell viability assays, live cell protease viability assays, tetrazolium reduction cell viability assays, resazurin reduction cell viability assays, dead-cell protease release cytotoxicity assays, lactate dehydrogenase release cytotoxicity assays, and DNA dye cytotoxicity assays. Other assays known in the art for measuring cell health, cell death and/or cell viability are also contemplated herein. Non-limiting examples of such assays follow.

Cell viability assays use a variety of markers as indicators of metabolically active (living) cells. Examples of markers commonly used include measuring ATP levels, measuring the ability to reduce a substrate, and detecting enzymatic/protease activities unique to living cells.

The RealTime-Glo™ MT Cell Viability Assay (Promega, Cat. #G9711) measures cell viability in real-time. In this assay, an engineered luciferase and a prosubstrate (which is not a substrate of luciferase) are added directly to the culture medium. The prosubstrate can penetrate cell membranes and enter cells. However, only viable cells with active metabolism can reduce the prosubstrate into a substrate for luciferase. The substrate then exits the cell where it is used by luciferase in the detection reagent to generate a luminescent signal. The same wells can be measured repeatedly for 3 days. The main advantages of this method are that it allows simple kinetic monitoring to determine dose response using fewer plates and cells. Also, because the method does not require cell lysis, the same cells can be used in additional cell-based assays or downstream applications.

ATP can be used to measure cell viability since only viable cells can synthesize ATP. ATP can be measured using the CellTiter-Glo® Luminescent Cell Viability Assay (Promega, Cat. #G7570) with reagents containing detergent, stabilized luciferase and luciferin substrate. The detergent lyses viable cells, releasing ATP into the medium. In the presence of ATP, luciferase uses luciferin to generate luminescence, which can be detected within 10 minutes using a luminometer. The CellTiter-Glo® 2.0 Assay (Promega, Cat. #G9241) is provided as a single solution that reduces reagent preparation time and provides the convenience of room temperature storage for easy implementation. These ATP assays do not require long incubation times to convert a substrate into a colored product. They also have excellent sensitivity and broad linearity, making them highly compatible with high-throughput applications where low cell numbers are used. They are also less prone to artifacts than other methods.

Live-cell protease activity disappears rapidly after cell death, so it is a useful marker of viable cells. Using the CellTiter-Fluor™ Cell Viability Assay (Promega, Cat. #G6080), live-cell protease activity can be measured using a cell-permeable fluorogenic protease substrate (GF-AFC). The substrate enters live cells where it is cleaved by live-cell protease to generate a fluorescent signal proportional to the number of viable cells. The incubation time for this method is 0.5-1 hour, which is shorter than tetrazolium assays (1-4 hours). Because this method does not lyse cells, it allows for multiplexing with many other assays in the same sample wells, including bioluminescent reporter cell-based assays.

Tetrazolium compounds used to detect viable cells fall into two basic categories:

Positively charged compounds (MTT) that readily penetrate viable cells:

Viable cells with active metabolism are able to convert MTT into a purple-colored formazan product. Thus, color formation can be a useful marker of viable cells. The CellTiter 96® Non-Radioactive Cell Proliferation Assay (MTT) (Promega, Cat. #G4000) uses this chemistry. However, the incubation time for this method is long (usually 4 hours). Also, the formazan product is insoluble, so a solubilizing reagent must be added prior to recording absorbance readings.

Negatively charged compounds (MTS, XTT. WST-1) that do not penetrate cells:

When using the CellTiter 96® AQueous One Solution Cell Proliferation Assay (MTS) (Promega, Cat. #G3582), negatively charged compounds must be combined with intermediate electron coupling reagents, which can enter cells, be reduced and then exit the cell to convert tetrazolium to the soluble formazan product. The incubation time for this method is 1-4 hours. There is no need to add a solubilizing reagent since the resulting formazan is soluble, making it more convenient.

Resazurin is a cell-permeable indicator dye that is dark blue in color with little intrinsic fluorescence. The CellTiter-Blue® Cell Viability Assay (Promega, Cat. #G8080) uses resazurin to measure cell viability. Only viable cells with active metabolism can reduce resazurin into resorufin, which is pink and fluorescent. After 1-4 hours of incubation, the signal is quantified using a microplate spectrophotometer or fluorometer. This method is relatively inexpensive and more sensitive than tetrazolium assays. However, fluorescence from compounds being tested may interfere with resorufin readings.

A disadvantage of all tetrazolium or resazurin reduction assays is that they depend on the accumulation of colored or fluorescent products over time. Since the signal gradually increases over time, a decrease in cell viability during this long incubation cannot be detected.

When cells die and lose membrane integrity, dead-cell proteases are released. A luminogenic substrate (CytoTox-Glo™ Cytotoxicity Assay, Promega, Cat. #G9290) or fluorogenic substrate (CytoTox-Fluor™ Cytotoxicity Assay, Promega, Cat. #G9260) can then be used to measure dead-cell protease activity. Because the substrate is not cell-permeable, essentially no signal from this substrate is generated by intact, viable cells. Furthermore, since the assays are non-lytic, they can be multiplexed with other compatible assay chemistries.

Dead cells that have lost membrane integrity release lactate dehydrogenase (LDH), which catalyzes the conversion of lactate to pyruvate with the concomitant production of NADH. Released LDH activity can be measured by providing excess substrates (lactate and NAD+) to produce NADH. This NADH can be measured using different assay chemistries:

1. LDH-Glo™ Cytotoxicity Assay: In the LDH-Glo™ Cytotoxicity Assay (Cat. #J2380), reductase uses NADH and reductase substrate (proluciferin) to generate luciferin. The luciferin is measured using a proprietary luciferase and the light signal is proportional to the amount of LDH, measured by a luminometer.

2. CytoTox-ONE™ Homogenous Membrane Integrity Assay: The CytoTox-ONE™ Homogeneous Membrane Integrity Assay (Promega, Cat. #G7890): Conversion of resazurin to a fluorescent resorufin product, measured using a fluorometer.

3. CytoTox 96® Non-Radioactive Cytotoxicity Assay: The CytoTox 96® Non-Radioactive Cytotoxicity Assay (Promega, Cat. #G1780) detects conversion of a tetrazolium salt (INT) into a red formazan product, measured by color absorbance

Some DNA-binding dyes are excluded from live cells but can enter and stain the DNA of permeable dead cells. Conventional dyes, like trypan blue, often require manual counting of stained cells using a hemocytometer, which is labor-intensive and not easily scalable. Another disadvantage of conventional dyes is they may be toxic to cells and can only be used for endpoint measurement.

Newer dyes, such as the CellTox™ Green Dye, produce a fluorescent signal when bound to DNA, which is easily measured using a fluorometer. It can be diluted in culture medium and delivered directly to cells at seeding or when treating with a test compound, allowing real-time kinetic measurement. The CellTox™ Green Cytotoxicity Assay (Promega, Cat. #G8741) is nontoxic, highly photo-stable and easily scalable.

All references, patents and patent applications disclosed herein are incorporated by reference with respect to the subject matter for which each is cited, which in some cases may encompass the entirety of the document.

The indefinite articles “a” and “an.” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean ±10% of the recited numerical value.

Where a range of values is provided, each value between and including the upper and lower ends of the range are specifically contemplated and described herein.

### EXAMPLES

### Example 1: Generation of Universal T Cell Agonist Protein MOKT3 and Lower Affinity Variants Thereof

A universal human T cell agonist (referred to herein as membrane-OKT3 (mOKT3) or pKSQ367) was generated by fusion of the following amino acid sequences:
a signal peptide from mouse IgG Heavy chain: MERHWIFLLLLSVTAGVHS (SEQ ID NO: 1);

a scFv from the mouse monoclonal anti-CD3e anti-
body clone OKT3:
(SEQ ID NO: 2)
QVQLQQSGAELARPGASVKMSCKASGYTFTRYTMHWVKQRPGQGLEWIGY
INPSRGYTNYNQKFKDKATLTTDKSSSTAYMQLSSLTSEDSAVYYCARYY
DDHYCLDYWGQGTTLTVSSGGGGSGGGGSGGGGSQIVLTQSPAIMSASPG
EKVTMTCSASSSVSYMNWYQQKSGTSPKRWIYDTSKLASGVPAHFRGSGS
GTSYSLTISGMEAEDAATYYCQQWSSNPFTFGSGTKLEIN;
and
the transmembrane domain from human CD8:
(SEQ ID NO: 12)
SHFVPVFLPAKPTTTPAPRPPTPAPTIASQPLSLRPEACRPAAGGAVHTR
GLDFACDIYIWAPLAGTCGVLLLSLVITLYCNHRNRRRVCKCPRPVVKSG
DKPSLSARYV.

A codon optimized cDNA encoding the mOKT3 protein was cloned in a lentiviral vector plasmid containing an EFIA promoter and followed by a T2A self-cleaving peptide (EGRGSLLTCGDVEENPGP (SEQ ID NO: 15)) and a blasticidin resistance gene (MAKPLSQEESTLIERATATINSIPISEDYSVASAALSSDGRIFTGVNVYHFTGGPCAEL VVLGTAAAAAAGNLTCIVAIGNENRGILSPCGRCRQVLLDLHPGIKAIVKDSDGQPT AVGIRELLPSGYVWEG* (SEQ ID NO: 16)). The final construct with the OKT3-CD8 protein expressed by EFIA promoter is also referred to herein as pKSQ367.

Lower affinity variants of the mOKT3 protein were constructed by site directed mutagenesis of the pKSQ367 lentiviral construct. Positions R55 and Y57 of the scFv were mutated to R55M and Y57A, respectively (known as mOKT3ma/pKSQ397), or R55L and Y57T, respectively (known as mOKT3lt/pKSQ398), as described in Chen et al. doi.org/10.3389/fimmu.2017.00793. The published affinities of these scFv variants are 250-fold lower (OKT3ma) or 1000-fold lower (OKT3lt) than the parental OKT3 clone (published affinity of KD of 5×10⁻¹⁰ M Law et al, Reinhertz et al.). The lower affinity variants of mOKT3 are to model more closely the affinities of natural T cell receptors (TCRs) with their recognized antigens (published affinities in the μM KD range). Many T cells will respond to the very strong stimulation of mOKT3, while lower affinity TCR binding allows the separation of subtle differences in TCR signaling thresholds from cell to cell and may allow more sensitive quality determinations between T cell therapy products prior to infusion into patients.

Lentiviral constructs were packaged by co-transfection of 293T cells with pCMV-VSV-G and psPax2 lentiviral packaging plasmids. Viral supernatant was transferred onto A375 cells and 48 hours after transduction the media was switched to blasticidin containing media to select for successfully transduced cells bearing the mOKT3 construct. The successfully transduced and selected A375 cells will be referred to as A375-pKSQ367 (also referred to as A375-mOKT3).

### Example 2: Generation of MOKT3-A375 Cells and Demonstration that they Activate T Cells

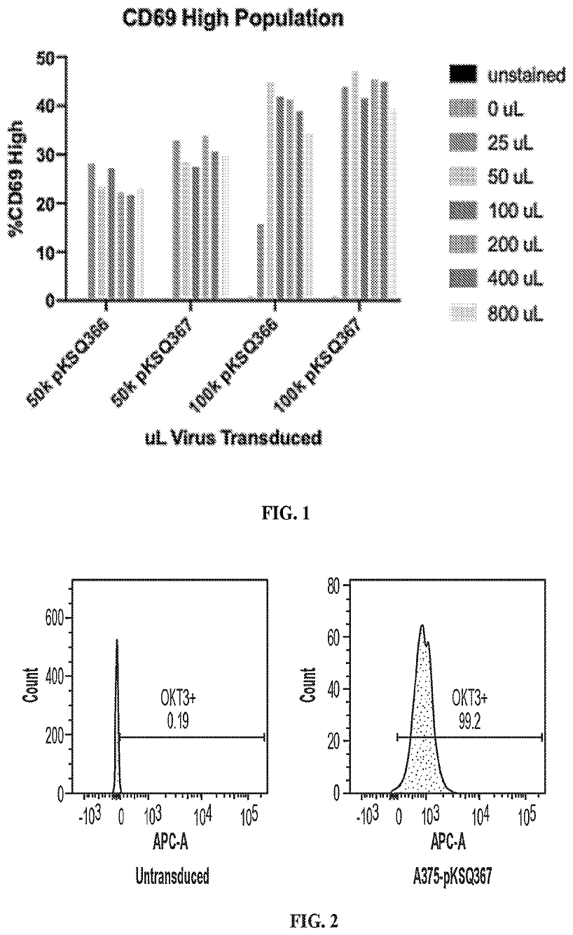

50,000 or 100,000 A375-pKSQ367 cells were plated per well of a 96-well plate in Dulbecco's Modified Eagle Medium (DMEM), fetal bovine serum (FBS), and an antibiotic. Pan T cells were isolated from peripheral blood mononuclear cells (PBMCs) and cocultured with the A375-pKSQ367 plated previously. Cocultures were performed at a 2:1 effector to target ratio (abbreviated as E:T). Media was removed from the A375-pKSQ367 plated previously, and 100,000 or 200,000 T cells in hematopoietic serum-free culture media with IL-2 were added to the wells containing 50,000 or 100,000 A375-pKSQ367, respectively. Supernatant was removed from the plate and cells were stained with anti-CD3 and anti-CD69 antibodies. Flow cytometry was performed, and CD69 expression was measured. CD69 activation was observed in all transduced samples (FIG. 1).

To verify surface expression of mOKT3, A375-pKSQ367 cells were incubated with goat serum in cell staining buffer before the cells were washed with cell staining buffer and then incubated with 1:100 goat-anti-mouse antibody. Cells were washed and flow cytometry was performed to assess expression. OKT3 expression was observed on 99.2% of transduced cells (FIG. 2).

Killing of mOKT3-A375 Cells by Tumor Infiltrating Lymphocytes (TILs)

To generate pre-REP TILs, melanoma tumor digest samples were thawed and plated at 1.5e6 cells/mL in pre-REP media with heat-inactivated Human AB serum, Pen/Step, HEPES buffer, Glutamax, beta-mercaptoethanol, gentamicin, IL-2, and DNase (added only to D277 and D3291 cultures). IL-2 was later added to the cells. The cells were counted and re-plated at 1e6 cells/mL in pre-REP media with IL-2 (D277 and D3291) or 1:1 pre-REP TIL media: hematopoietic serum-free culture media and IL-2 (D5746). Re-plating and feeding of IL-2 was repeated until growth slowed down to less than 1.5× growth after two days. D277, D3291, and D5746 were frozen down after the culture period.

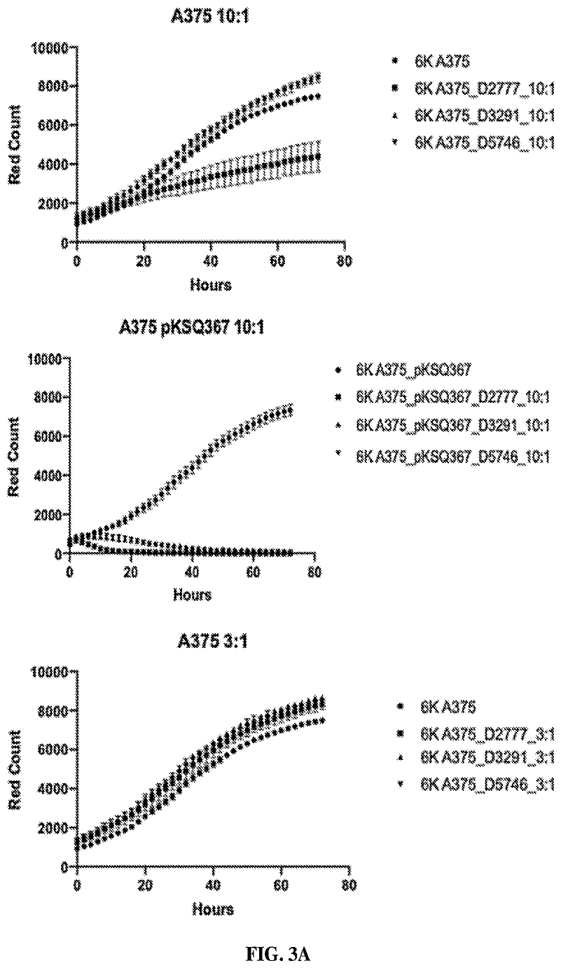

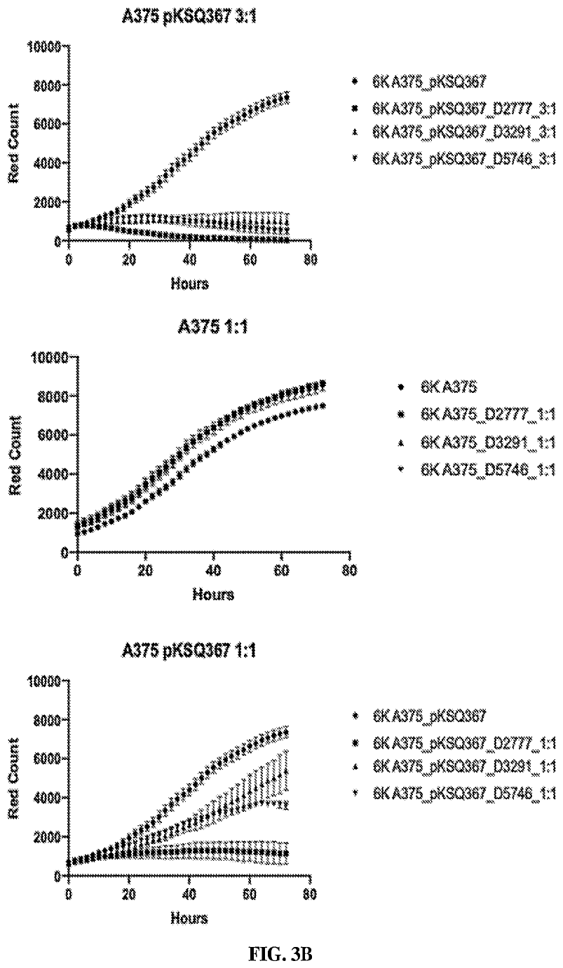

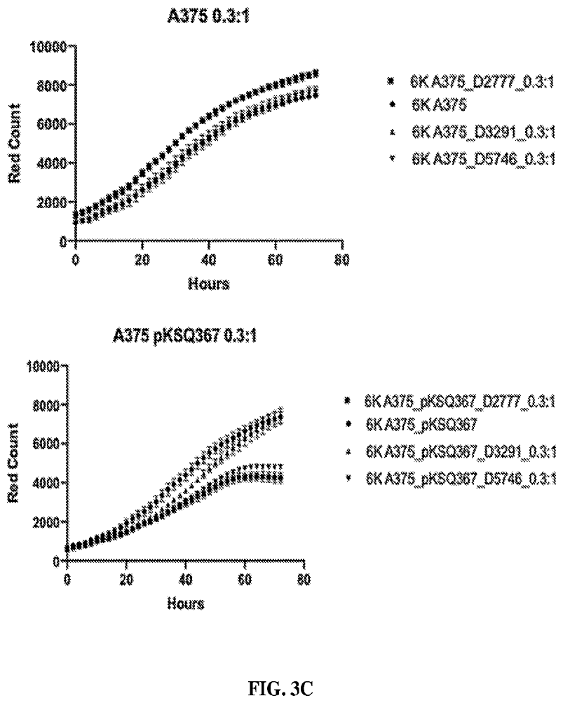

The day before coculture initiation, pre-REP TILs were thawed from three donors and rested overnight in REP media with IL-2. 6,000 A375-pKSQ367 were plated in a 96-well plate the day before coculture initiation in REP media with IL-2 and Caspase 3/7 dye. The following day media was removed from the 96-well plate and TILs were added to the A375-pKSQ367 that were plated the previous day at a 10:1, 3:1, 1:1, or 0.3:1 E:T). The plate was imaged every 2 hours for 3 days by the IncuCyte. TIL recognition and killing of A375-pKSQ367 cells was observed across all E:T tested (FIGS. 3A-3C).

### Example of Alternative OKT3 Membrane Anchors

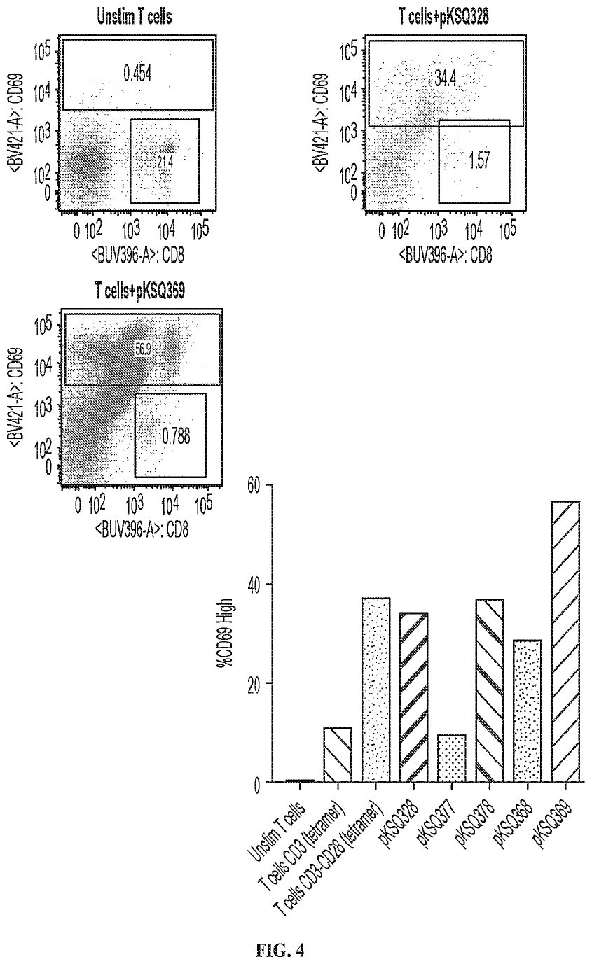

The objective of this experiment was to show that various signal peptides and membrane anchors may be used to express mOTK3 on the cell surface, and that mOKT3 can be expressed by other cell types in addition to A375 cells. 2 million K562 cells were transduced with lentivirus encoding for pKSQ328, pKSQ377, pKSQ378, pKSQ368, and pKSQ369 constructs containing OKT3 scFv fused to a variety of signal peptides such as mouse IgG, human CSF2R and human CD5, and membrane anchor proteins including mouse CD8a, human CD8a, human CD28 and human CD14. 48 hours after transduction the transduced K562 cells were selected for blasticidin resistance marker if present. On day 8 post transduction, K562 stable cell lines were plated at 100,00 cells/well in the presence of 100,000 pan T cells isolated from donor PBMCs in IL-2 containing medium. 24 hours after plating, cells were stained with anti CD8 and anti-CD69 antibodies. Flow cytometry was performed and CD8+ T cells were quantified for surface expression of CD69 (a marker of T cell activation) relative to unstimulated controls. We observed that multiple combinations of signal peptides and membrane anchors used to construct mOKT3 constructs drove activation of T cells (FIG. 4).

### Example 3: Characterization: Expression of MOKT3, Coculture with Pan T Cells and CD69 Induction

A375 parental cells (containing NucLightRed, a red fluorescence nuclear reporter) were transduced with pKSQ367, pKSQ396, pKSQ397 or pKSQ398. The ‘low affinity’ constructs, pKSQ396, pKSQ397 and pKSQ398, were transduced with three different volumes of virus to ensure transduction.

To assess efficacy of transduction and constructs, several assays were performed.

1. Expression of mOKT3 on the cell surface.

2. Activation of pan T cells though their expression of CD69 after coculture with A375 transduced target cells.

3. Reduction in growth (an indicator of recognition by TIL) of A375 transduced cells after coculture with patient derived melanoma TIL.

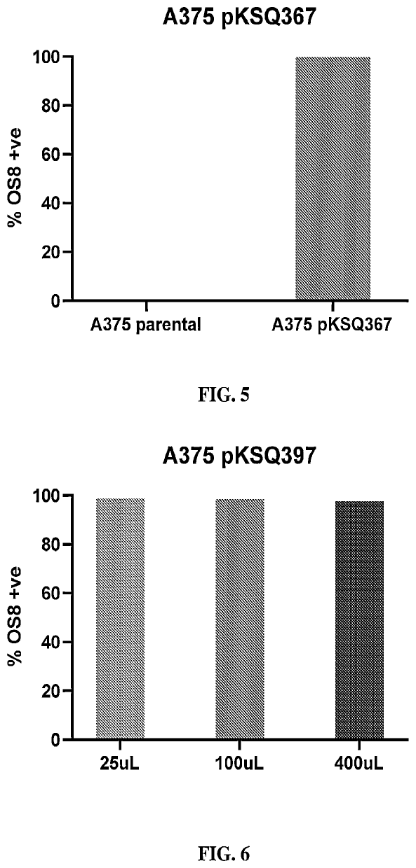

A375 cells (both A375 parental line and the transduced lines) were resuspended at 1.5e6/mL cell culture media then plated at 0.75e5/50 μL/well in a 96 well V bottom plate and incubated overnight. The next day, plated cells were blocked by Goat Serum in Cell Staining Buffer and incubated in the dark. Subsequently, the cells were stained by (1:100) goat anti-mouse IgG2a polyclonal, Alexa647 in Cell Staining Buffer and incubated. After washing, the cells were resuspended in Cell Staining Buffer and acquired on BD Fortessa. (FIGS. 5-7)

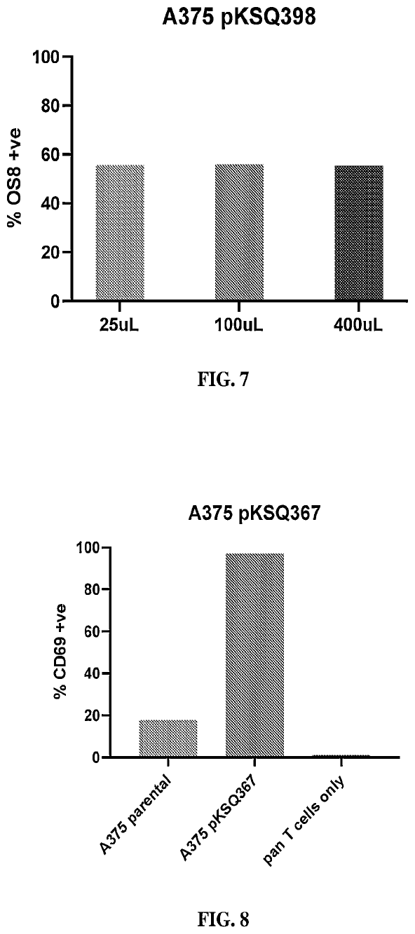

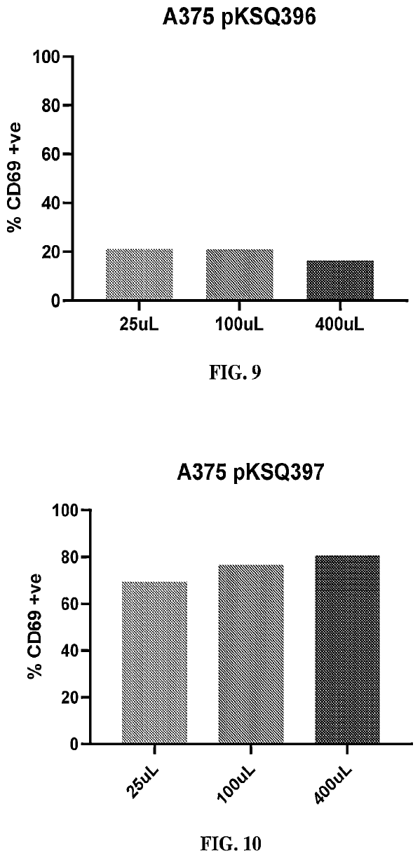

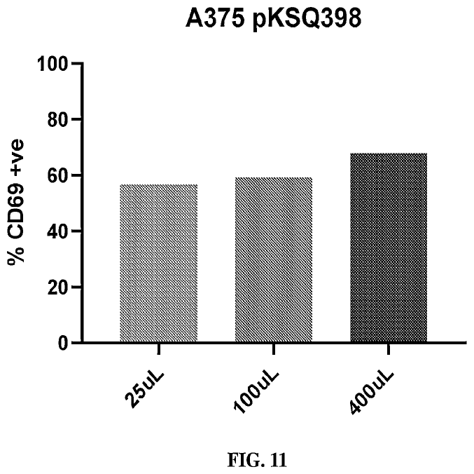

A375 cells (both A375 parental line and the transduced lines) were resuspended at 1.5e6/mL in cell culture media then plated at 0.75e5/50 μL/well in a 96 well V bottom plate and incubated overnight. Following day; PBMCs (Donor #148192) was thawed, pan T cells were isolated following the manufacturer protocol. Isolated Pan T cells were counted and resuspended at 1.5e6/mL. 0.75e5 isolated pan T cells were added (50 μL per well) to the plated A375 target cells and incubated overnight. The plated cells were washed by Cell Staining Buffer, stained for anti-CD69 Ab BV785 in Cell Staining Buffer and incubated. After washing, the cells were resuspended in Cell Staining Buffer and acquired on BD Fortessa. (FIGS. 8-11)

### Example 4: Two-Dimensional A375-PKSQ367 Coculture Model

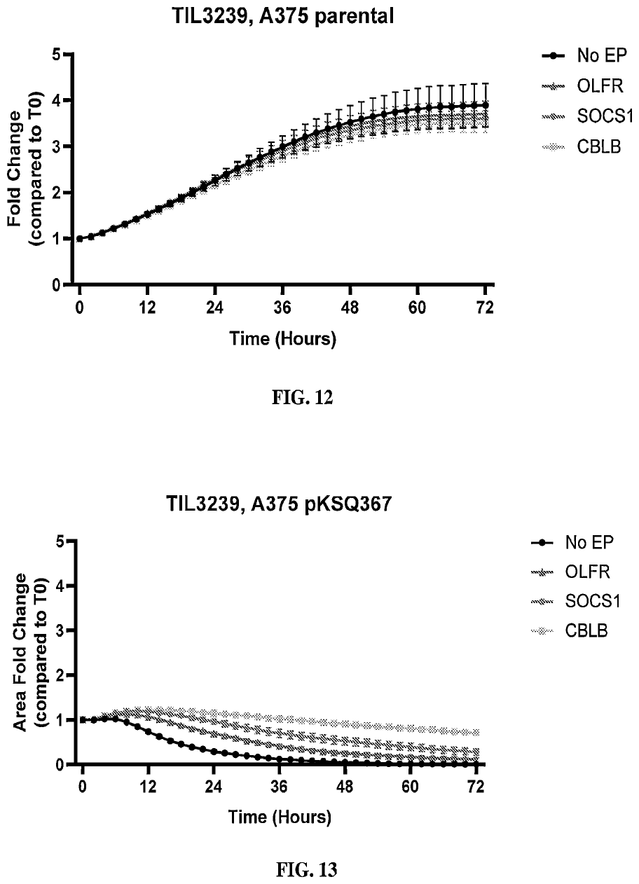

A375 cells (both A375 parental line and the transduced lines) were resuspended at 1.2e5/mL in cell culture media then plated at 6e3/50u L/well in a 96-Well flat bottom plate and incubated overnight. Following day; 6e3/50u L/well of each TIL condition in REP media (AIMV, RPMI, Human AB Serum) was added on top of the plated A375 target cells. 1 REP media containing IL-2 was added to each coculture well. The coculture plate was incubated at room temperature before moving into the IncuCyte. The coculture plate was incubated in the IncuCyte for an additional period of time before beginning image collection. An image was acquired every 2 hours for 72 hours total (Read Phase & Red at 10× objective). IncuCyte images were quantified on total area of red fluorescence detected in each well, then normalized back to 0 hr to identify changes in target cell expansion/reduction over time. (FIGS. 12-14)

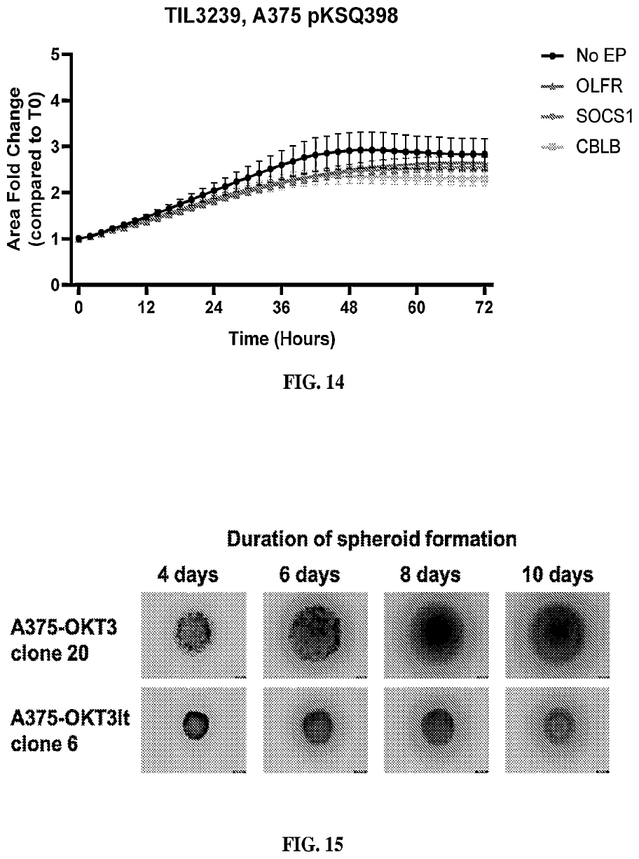

In FIGS. 13 and 14, A375 pKSQ367 and A375 pKSQ398 cells were each cultured as a monolayer. Unedited and gene edited TILs were added into the culture and TIL cytotoxicity was measured by changes in tumor cell area through imaging. In the case of A375 pKSQ367 as tumor target, neither SOCS1 nor CBLB gene edited TILs showed better control of tumor than unedited TILs (FIG. 13); in the case of A375 pKSQ398 as tumor target, all of the TILs, whether edited or unedited, were unable to control tumor growth (FIG. 14). Thus, in the setting in which the tumor targets are cultured as monolayers, the efficacy of unedited vs edited TILs cannot be differentiated.

### Example 5: Three-Dimensional A375-PKSQ367 Spheroid Coculture Model

Using A375-pKSQ367 spheroids as target cells for coculture assays was explored, as the 3D tumor spheroid may provide a more challenging target and complex microenvironment than a monolayer of target cells. This could enable us to see subtle potency differences between edited and non-edited TILs.

Both high-affinity (pKSQ367) and low-affinity (pKSQ398) A375 lines generated from single clones were used for spheroid-based cocultures.

A375-pKSQ367 high- and low-affinity clones were maintained in DMEM supplemented with heat-inactivated FBS and an antibiotic. A375-pKSQ367 high affinity single cell clone 20 was used for high-affinity spheroid coculture assays, A375-pKSQ367 low affinity single cell clone 6 was used for low-affinity spheroid coculture assays.

For A375-pKSQ367 spheroid cocultures, a common protocol was followed:
Day −4: A375-pKSQ367 were passaged into RPMI supplemented with FBS and antibiotic and plated at a density of 5,000-10,000 live cells/well. Plates were transferred to the Incucyte imaging system inside a 37° C. 5% CO₂ incubator and incubated to facilitate spheroid formation. Images were taken at 4× magnification in the phase and red channels once every few hours.
Day −1: cryopreserved TIL from 4 donors (Donor 1: D4267, NSCLC, Donor 2: D4397. NSCLC. Donor 3: D6164, Melanoma, Donor 4: D6481, Melanoma) was thawed, washed, counted, and resuspended at 3e\hat{ }6 live cells per mL in TIL media (RPMI 1640, AIM V, supplemented with human AB serum). IL-2 was added. Cells were rested in appropriately sized flasks overnight in a 37° C. 5% CO₂ incubator.
Day 0: TIL were filtered over a cell strainer into an appropriately sized conical tube, centrifuged, and counted. Plate containing A375-pKSQ367 spheroids was removed from the Incucyte. TIL were added to A375-pKSQ367 spheroids at effector cell to target cell (E:T) ratios from 10:1 to 0.625:1. E:T ratios were calculated based on live TIL (effector) counts on Day 0 and A375-pKSQ367 (target) initial seeding density. Some wells did not have TIL added and served as growth controls. Plates were transferred to the Incucyte imaging system inside a 37° C. 5% CO₂ incubator and imaging was continued as on Day −4.
Day 6: imaging was stopped and analysis was conducted by visual assessment of images collected by the Incucyte.

In this case, analysis was conducted by converting the images to black and white and comparing the area of the spheroid (black) remaining in a well at a given time point. This data was also analyzed by assessing the intensity of the spheroid's fluorescence. A reduction in fluorescence level was used as an indicator of spheroid death. This assay could also be designed to assess target cell apoptosis by reading out by chromium release or caspase 3 expression.

The morphology of A375-pKSQ367 spheroids was assessed in wells where no TIL was added. Differences in high- and low-affinity spheroid morphology were observed. Specifically, at the same plating density (10,000 cells/well), the high-affinity line (labeled as “A375-pKSQ367” in the figure) formed a much “larger”/“looser” spheroid than the low-affinity line (labeled as “A375-OKT3lt” in the figure). Morphological differences were observable throughout the duration of the spheroid coculture (days 4-10 after A375-OKT3 seeding). It is possible that differences in morphology also exist between clones of the same lineage, and that these differences in morphology could offer another opportunity to tease out subtle differences between T cells. (FIG. 15)

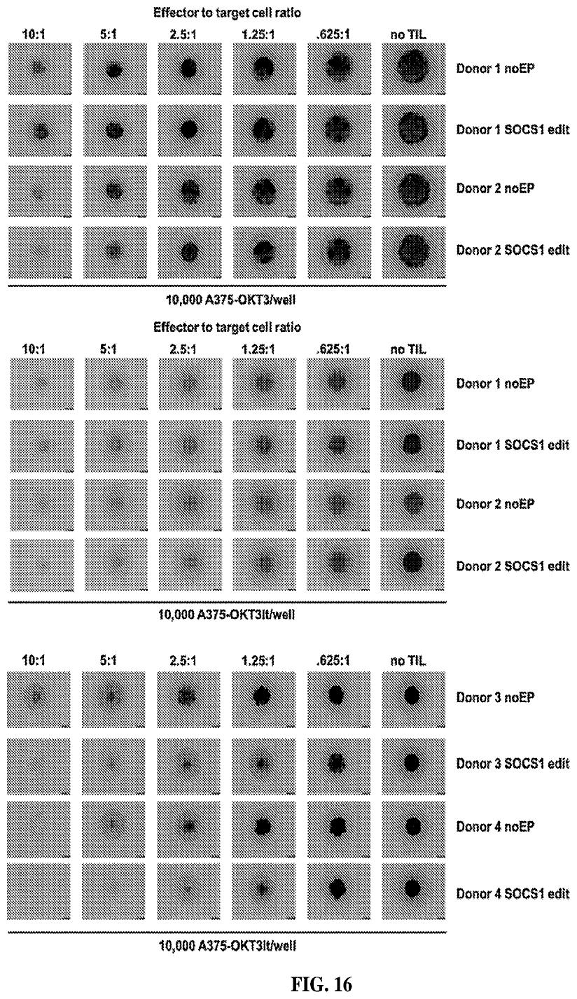

The cytotoxicity of TILs against A375-pKSQ367 spheroids was assessed by comparing the effect of increased ET ratio at the same time point. When TIL and A375-pKSQ367 spheroids were cocultured, dose-dependent killing by both noEP and SOCS1-edited TIL was observed against both high-affinity (labeled as “A375-OKT3” in the figure) (Donors 1 and 2) and low affinity (labeled as “A375-OKT3It” in the figure) (Donors 1, 2, 3, and 4) spheroids. (FIG. 16)

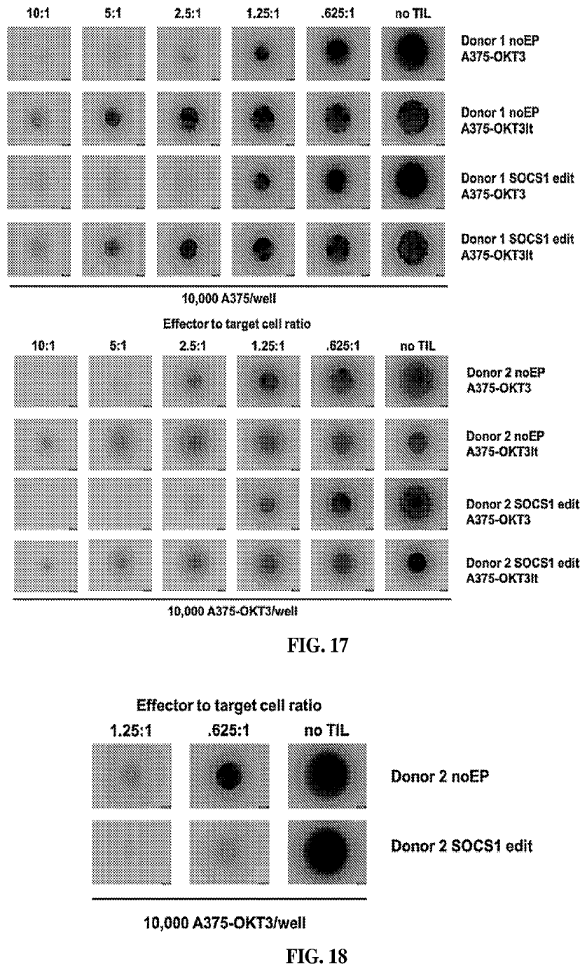

The difference in cytotoxicity dynamics of TIL against A375-pKSQ367 high-(labeled as “A375-pKSQ367” in the figure) and low-affinity (labeled as “A375-pKSQ367A375-pKSQ398” in the figure) spheroids was assessed by comparing the size of high- and low-affinity spheroids remaining at the same time point. We observed that the high affinity spheroid was killed more quickly than the low affinity spheroid by donors 1 and 2. (FIG. 17)

In FIG. 17, in which A375 pKSQ367 (A375-OKT3) tumor cells were growth as a 3D tumor spheroid for 4 days prior to TIL addition, SOCS1 edited TIL demonstrated greater ability to kill the tumor than the unedited control (No EP), with complete clearance of the tumor by SOCS1 edited TILs at 1.25:1 ratio while residual tumors are seen in the well with unedited control. Enhanced efficacy of SOCS1 edited TILs was also observed when A375 pKSQ398 (A375-OKT3lt) cells were grown as 3D tumor spheroids, although the difference is observed at the higher E:T ratios (5:1).

Whether SOCS1-edited TIL drove increased killing of high-affinity A375-pKSQ367 spheroids at the same E:T was assessed. Increased cytotoxicity against high affinity A375-pKSQ367 spheroids by SOCS1-edited TIL was observed for Donor 2. These images were captured after 5 days of coculture, demonstrating that A375-pKSQ367 can distinguish between TIL of different anti-tumor potencies. (FIG. 18)

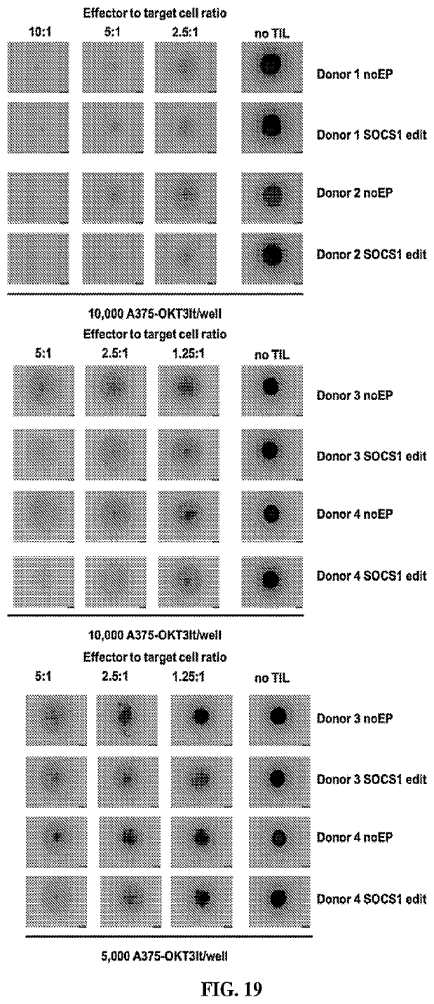

Whether SOCS1-edited TIL drove increased killing of low-affinity A375-pKSQ367 spheroids was assessed. For Donors 2, 3, and 4, we observed increased potency against A375-pKSQ367 low-affinity spheroids from SOCS1-edited TIL. These results demonstrate that A375-pKSQ367 low-affinity spheroids increase the sensitivity of distinguishing between TIL populations of different potencies (FIG. 19)

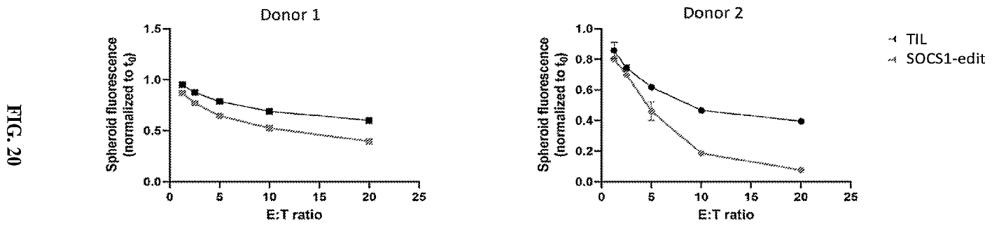

Whether SOCS1-edited TIL drove increased killing of low-affinity A375-pKSQ367 spheroids was assessed by spheroid fluorescence. A375-OKT3lt cells were plated in 96-well ultra-low attachment plates and cultured for 4 days to allow for spheroid formation. On day 4, SOCS1-edited TIL or control (e.g., unedited) TIL were thawed and added to the spheroid cultures at various Effector: Target (E:T) ratios. Cytotoxicity of spheroids by SOCS1-edited TIL or control (e.g., unedited) TIL was monitored by InCucyte as a function of changes in spheroid fluorescence level. SOCS1-edited TIL exhibited increasing killing of low-affinity A375-pKSQ367 spheroids with increasing E:T ratios (FIG. 20)

### Example 6: Use the A375-PKSQ367 Model to Evaluate TIL Potency Based on IFN-γ and/or IL-6 Cytokine Release

TIL potency can also be measured from supernatants based on cytotoxicity and/or cytokine release. Cytotoxicity and effector cytokine release were evaluated using low-affinity A375-pKSQ367 cells three-dimensional (3D) spheroids as target cells for coculture assays. High-(pKSQ367) lines generated from single clones could also be used as described below.

A375-pKSQ367 high- and low-affinity clones were maintained in DMEM supplemented with heat-inactivated FBS and antibiotic for single-cell suspension and 3D spheroid coculture assays.

For A375-pKSQ367 3D spheroid cocultures, a similar protocol was followed:
Day −4 (for 3D spheroid co-cultures only): A375-pKSQ367 were passaged into RPMI supplemented with FBS and antibiotic and plated at a density of 5,000-10,000 live cells/well. Plates were transferred to the Incucyte imaging system inside a 37° C. 5% CO₂ incubator and incubated to facilitate spheroid formation. Images are taken at 4× magnification in the phase and red channels once every few hours.
Day −1: cryopreserved TIL were thawed, washed once, counted, and resuspended at 3e6 live cells per mL in TIL media (a 1:1 mixture of RPMI 1640 and AIM V, supplemented with human AB serum). IL-2 is added. Cells were rested in appropriately sized flasks overnight in a 37° C. 5% CO₂ incubator. For single-cell suspension assays, A375-pKSQ367 cells were plated at a density of 5,000-10,000 live cells/well. Plates were transferred inside a 37° C. 5% CO₂ incubator overnight prior to addition of TIL.
Day 0: TIL were filtered over a cell strainer into an appropriately sized conical tube, centrifuged, and counted. Plate containing A375-pKSQ367 spheroids and/or A375-pKSQ367 single-cell suspensions are removed from the incubator. TIL were added at effector cell to target cell (E:T) ratios from 20:1 to 0.625:1. E:T ratios were calculated based on live TIL (effector) counts on Day 0 and A375-pKSQ367 (target) initial seeding density. Some wells did not have TIL added and served as growth controls. Plates were transferred to a 37° C. 5% CO₂ incubator or to an Incucyte imaging system inside a 37° C. 5% CO₂ incubator as appropriate for the assay, and imaging continued.
3D Spheroid Assay: Between Day 1-Day 2 following addition of TIL, imaging was stopped and analysis conducted by visual assessment of images collected by the Incucyte. The cytotoxicity of TILs against A375-pKSQ367 spheroids was assessed by comparing the effect of increased E:T ratio at this time point, with supernatants harvested in parallel. The presence of IFNγ and/or IL-6 production and other cytokines was detected directly in the supernatants. As an alternative approach to assessing TIL cytotoxicity, the level of lactate dehydrogenase, which is released rapidly into the supernatant upon target cell lysis, was also measured in the supernatant using the LDH-Glo™ Cytotoxicity Assay (Promega).

TILs with increased potency were predicted to produce higher amounts of IFN□ and IL-6 cytokine production, as evaluated by ELISA, MSD or Luminex assay, in either a single-cell suspension or spheroid co-culture assay, and/or show greater cytotoxicity as reflected in increased measurement of lactate dehydrogenase in the supernatant.

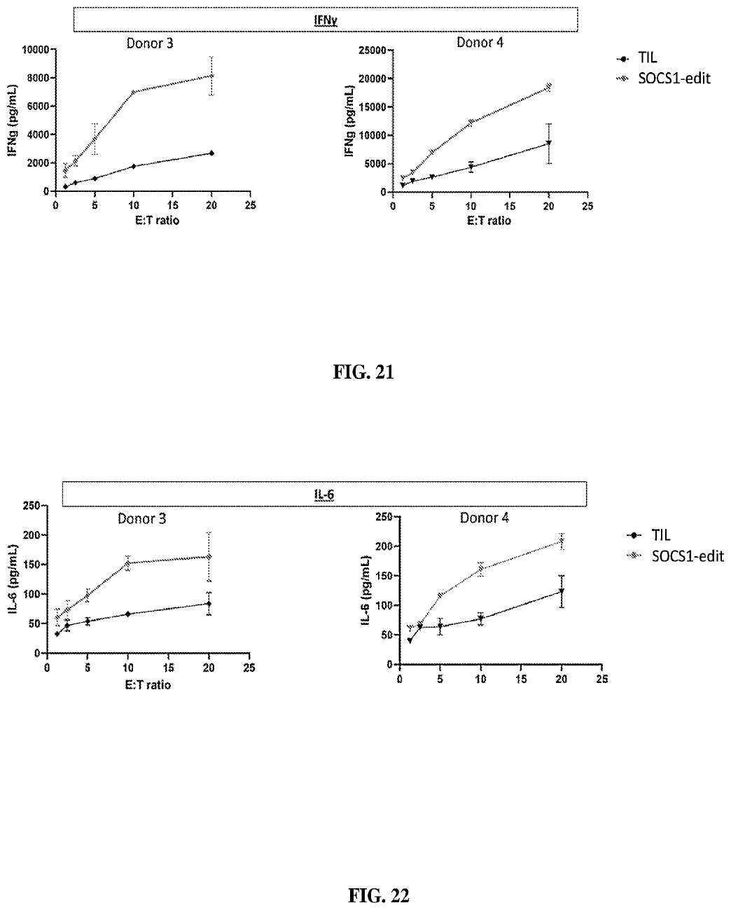

Whether SOCS1-edited TIL grown in coculture with low-affinity A375-pKSQ367 spheroids had increased potency of IFNγ and/or IL-6 was assessed. Increasing E:T ratios of SOCS1-edited TIL cocultured with low-affinity A375-pKSQ367 spheroids exhibited increasing secretion of both IFNγ (FIG. 21) and IL-6 (FIG. 22). SOCS1 edited TIL produced higher levels of both cytokines across all E:T ratios.

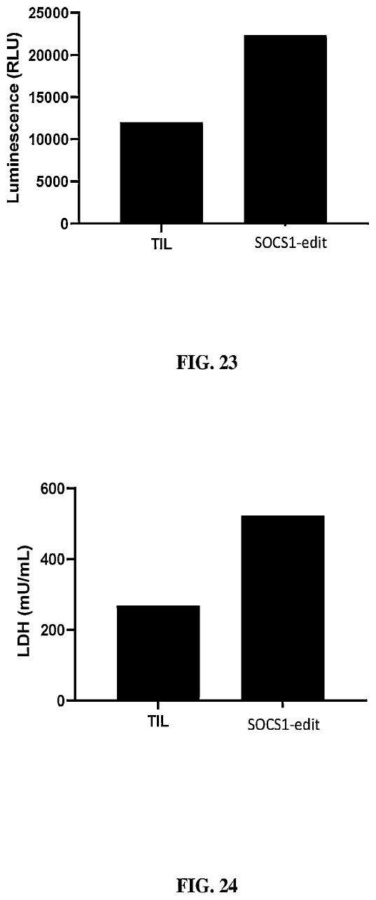

Supernatants from co-cultures of SOCS1-edited TIL and low affinity A375-pKSQ367 co-cultures also showed higher levels of lactate dehydrogenase in comparison to co-cultures with control TIL, consistent with enhanced cytotoxicity leading to greater amount of A375-KSQ367 cell death and lactate dehydrogenase release (FIG. 23 and FIG. 24)

TABLE 1
Sequences relevant to the disclosure

| | | | | | Resistance |
|---|---|---|---|---|---|
| | Promoter | signal peptide | CD3 binder | Membrane anchor | marker |
| PKSQ328: | EF1A | Mouse IGG heavy | OKT3 scFv: | Mouse CD8a amino | GFP |
| Mouse IgG signal | | chain: | QVQLQQSGAELARPGAS | acids 100-207: | |
| peptide and mouse cd8 | | MERHWIFLLLLS | VKMSCKASGYTFTRYTM | SSVVPVLQKVNSTTTKPV | |
| transmembrane domain | | VTAGVHS (SEQ | HWVKQRPGQGLEWIGYI | LRTPSPVHPTGTSQPQRP | |
| | | ID NO: 1) | NPSRGYTNYNQKFKDKA | EDCRPRGSVKGTGLDFAC | |
| | | | TLTTDKSSSTAYMQLSS | DIYIWAPLAGICVALLLS | |
| | | | LTSEDSAVYYCARYYDD | LIITLICYHRSRKRVCKC | |
| | | | HYCLDYWGQGTTLTVSS | PRPLVRQEGKPRPSEKIV | |
| | | | GGGGSGGGGSGGGGSQI | (SEQ ID NO: 3) | |
| | | | VLTQSPAIMSASPGEKV | | |
| | | | TMTCSASSSVSYMNWYQ | | |
| | | | QKSGTSPKRWIYDTSKL | | |
| | | | ASGVPAHFRGSGSGTSY | | |
| | | | SLTISGMEAEDAATYYC | | |
| | | | QQWSSNPFTFGSGTKLE | | |
| | | | IN (SEQ ID NO: 2) | | |
| PKSQ377 | EF1A | CSF2RA: | OKT3 scFv: | human CD8a aa138-235: | Blasticidin |
| Human CSF2R signal | | MLLLVTSLLLCE | QVQLLESGAELARPGAS | TTTPAPRPPTPAPTIASQ | |
| peptide, OKT3 scFv | | LPHPAFLLIP | VKMSCKASGYTFTRYTM | PLSLRPEACRPAAGGAVH | |
| with glycine-serine | | (SEQ ID NO: 4) | HWVKQRPGQGLEWIGYI | TRGLDFACDIYIWAPLAG | |
| linker, and human cd8 | | | NPSRGYTNYNQKFKDKA | TCGVLLLSLVITLYCNHR | |
| transmembrane domain | | | TLTTDKSSSTAYMQLSS | NRRRVCKCPRPVVKSGDK | |
| | | | LTSEDSAVYYCAGYYDD | PSLSARYVN (SEQ ID | |
| | | | HYCLDYWGQGTLVTVSS | NO: 6) | |
| | | | GGGGSGGGGSGGGGSDI | | |
| | | | VMTQSPAIMSASPGEKV | | |
| | | | TMTCSASSSVSYMNWYQ | | |
| | | | QKSGTSPKRWIYDTSKL | | |
| | | | ASGVPAHFRGSGSGTSY | | |
| | | | SLTISGMEAEDAATYYC | | |
| | | | QQWSSNPFTFGSGTKLE | | |
| | | | IKRTTT (SEQ ID NO: | | |
| | | | 5) | | |
| PKSQ378: | EF1A | CSF2RA: | OKT3 scFv: | human CD8a aa138-235: | Blasticidin |
| human CSF2RA signal | | MLLLVTSLLLCE | QVQLQQSGAELARPGAS | TTTPAPRPPTPAPTIASQ | |
| peptide, non-glycine- | | LPHPAFLLIP | VKMSCKASGYTFTRYTM | PLSLRPEACRPAAGGAVH | |
| serine linker for OKT3 | | (SEQ ID NO: 4) | HWVKQRPGQGLEWIGYI | TRGLDFACDIYIWAPLAG | |
| scFv fused to human | | | NPSRGYTNYNQKFKDKA | TCGVLLLSLVITLYCNHR | |
| cd8 transmembrane | | | TLTTDKSSSTAYMQLSS | NRRRVCKCPRPVVKSGDK | |
| domain | | | LTSEDSAVYYCARYYDD | PSLSARYVN (SEQ ID | |
| | | | HYCLDYWGQGTTLTVSS | NO: 6) | |
| | | | AKTTPKLEEGEFSEARV | | |
| | | | DIVLTQSPAIMSASPGE | | |
| | | | KVTMTCSASSSVSYMNW | | |
| | | | YQQKSGTSPKRWIYDTS | | |
| | | | KLASGVPAHFRGSGSGT | | |
| | | | SYSLTISGMEAEDAATY | | |
| | | | YCQQWSSNPFTFGSGTK | | |
| | | | LEINR (SEQ ID NO: 7) | | |
| PKSQ368: | SFFV | CD5: | OKT3 scFv: | human CD28 | GFP |
| Human CD5 signal | | MPMGSLQPLATL | VLAQVQLQQSGAELARP | transmembrane: | |
| peptide, glycine-serine | | YLLGMLVAS | GASVKMSCKASGYTFTR | PSPLFPGPSKPFWVLVV | |
| linker OKT3 scFv | | (SEQ ID NO: 8) | YTMHWVKQRPGQGLEWI | VGGVLACYSLLVTVAF | |
| fused to human CD28 | | | GYINPSRGYTNYNQKFK | IIFWVRSKRSRLLHSDY | |
| transmembrane domain | | | DKATLTTDKSSSTAYMQ | MNMTPRRPGPTRKHY | |
| | | | LSSLTSEDSAVYYCARY | QPYAPPRDFAAYRS | |
| | | | YDDHYCLDYWGQGTTVT | (SEQ ID NO: 10) | |
| | | | VSAGGGGSGGGGSGGGS | | |
| | | | SQIVLTQSPAIMSASPG | | |
| | | | EKVTMTCSASSSVSYMN | | |
| | | | WYQQKSGTSPKRWIYDT | | |
| | | | SKLASGVPAHFRGSGSG | | |
| | | | TSYSLTISGMEAEDAAT | | |
| | | | YYCQQWSSNPFTFGSGT | | |
| | | | KLEINRGGGDP (SEQ | | |
| | | | ID NO: 9) | | |
| PKSQ369: | SFFV | CD5: | OKT3 scFv: | human CD14: | GFP |
| Human CD5 signal | | MPMGSLQPLATL | VLAQVQLQQSGAELARP | TTPEPCELDDEDFRCVCN | |
| peptide, glycine-serine | | YLLGMLVAS | GASVKMSCKASGYTFTR | FSEPQPDWSEAFQCVSAV | |
| linker OKT3 scFv, | | (SEQ ID NO: 8) | YTMHWVKQRPGQGLEWI | EVEIHAGGLNLEPFLKRV | |
| fused to human CD14 | | | GYINPSRGYTNYNQKFK | DADADPRQYADTVKALRV | |
| protein | | | DKATLTTDKSSSTAYMQ | RRLTVGAAQVPAQLLVGA | |
| | | | LSSLTSEDSAVYYCARY | LRVLAYSRLKELTLEDLK | |
| | | | YDDHYCLDYWGQGTTVT | ITGTMPPLPLEATGLALS | |
| | | | VSAGGGGSGGGGSGGGS | SLRLRNVSWATGRSWLAE | |
| | | | SQIVLTQSPAIMSASPG | LQQWLKPGLKVLSIAQAH | |
| | | | EKVTMTCSASSSVSYMN | SPAFSCEQVRAFPALTSL | |
| | | | WYQQKSGTSPKRWIYDT | DLSDNPGLGERGLMAALC | |
| | | | SKLASGVPAHFRGSGSG | PHKFPAIQNLALRNTGME | |
| | | | TSYSLTISGMEAEDAAT | TPTGVCAALAAAGVQPHS | |
| | | | YYCQQWSSNPFTFGSGT | LDLSHNSLRATVNPSAPR | |
| | | | KLEINRGGGDP (SEQ | CMWSSALNSLNLSFAGLE | |
| | | | ID NO: 9) | QVPKGLPAKLRVLDLSCN | |
| | | | | RLNRAPQPDELPEVDNLT | |
| | | | | LDGNPFLVPGTALPHEGS | |
| | | | | MNSGVVPACARSTLSVGV | |
| | | | | SGTLVLLQGARGFA | |
| | | | | (SEQ ID NO: 11) | |
| PKSQ366 | EF1A | Mouse IGG heavy | OKT3 scFv: | Mouse CD8a amino | Blasticidin |
| Mouse IgG heavy | | chain: | QVQLQQSGAELARPGAS | acids 100-207: | |
| chain signal peptide, | | MERHWIFLLLLS | VKMSCKASGYTFTRYTM | SSVVPVLQKVNSTTTKPV | |
| OKT3 scFv with | | VTAGVHS (SEQ | HWVKQRPGQGLEWIGYI | LRTPSPVHPTGTSQPQRP | |
| glycine-serine linker | | ID NO: 1) | NPSRGYTNYNQKFKDKA | EDCRPRGSVKGTGLDFAC | |
| fused to mouse CD8 | | | TLTTDKSSSTAYMQLSS | DIYIWAPLAGICVALLLS | |
| transmembrane domain | | | LTSEDSAVYYCARYYDD | LIITLICYHRSRKRVCKC | |
| | | | HYCLDYWGQGTTLTVSS | PRPLVRQEGKPRPSEKIV | |
| | | | GGGGSGGGGSGGGGSQI | (SEQ ID NO: 3) | |
| | | | VLTQSPAIMSASPGEKV | | |
| | | | TMTCSASSSVSYMNWYQ | | |
| | | | QKSGTSPKRWIYDTSKL | | |
| | | | ASGVPAHFRGSGSGTSY | | |
| | | | SLTISGMEAEDAATYYC | | |
| | | | QQWSSNPFTFGSGTKLE | | |
| | | | IN (SEQ ID NO: 2) | | |
| PKSQ367: | EF1A | Mouse IGG heavy | OKT3 scFv: | human CD8a aa 1-235: | Blasticidin |
| Mouse IGG signal | | chain: | QVQLQQSGAELARPGAS | SHFVPVFLPAKPTTTPAP | |
| peptide, OKT3 scFv | | MERHWIFLLLLS | VKMSCKASGYTFTRYTM | RPPTPAPTIASQPLSLRP | |
| with glycine-serine | | VTAGVHS (SEQ | HWVKQRPGQGLEWIGYI | EACRPAAGGAVHTRGLDF | |
| linker fused to entire | | ID NO: 1) | NPSRGYTNYNQKFKDKA | ACDIYIWAPLAGTCGVLL | |
| human CD8 sequence | | | TLTTDKSSSTAYMQLSS | LSLVITLYCNHRNRRRVC | |
| | | | LTSEDSAVYYCARYYDD | KCPRPVVKSGDKPSLSAR | |
| | | | HYCLDYWGQGTTLTVSS | YV (SEQ ID NO: 12) | |
| | | | GGGGSGGGGSGGGGSQI | | |
| | | | VLTQSPAIMSASPGEKV | | |
| | | | TMTCSASSSVSYMNWYQ | | |
| | | | QKSGTSPKRWIYDTSKL | | |
| | | | ASGVPAHFRGSGSGTSY | | |
| | | | SLTISGMEAEDAATYYC | | |
| | | | QQWSSNPFTFGSGTKLE | | |
| | | | IN (SEQ ID NO: 2) | | |
| PKSQ397: | EF1A | Mouse IGG heavy | OKT3ma scFv: | human CD8a aa 1-235: | Blasticidin |
| Mouse IgG signal | | chain: | MERHWIFLLLLSVTAGV | SHFVPVFLPAKPTTTPAP | |
| peptide, OKTma lower | | MERHWIFLLLLS | HSQVQLQQSGAELARPG | RPPTPAPTIASQPLSLRP | |
| affinity scFv with | | VTAGVHS (SEQ | ASVKMSCKASGYTFTRY | EACRPAAGGAVHTRGLDF | |
| glycine-serine linker | | ID NO: 1) | TMHWVKQRPGQGLEWIG | ACDIYIWAPLAGTCGVLL | |
| fused to human CD8 | | | YINPSMGATNYNQKFKD | LSLVITLYCNHRNRRRVC | |
| | | | KATLTTDKSSSTAYMQL | KCPRPVVKSGDKPSLSAR | |
| | | | SSLTSEDSAVYYCARYY | YV (SEQ ID NO: 12) | |
| | | | DDHYCLDYWGQGTTLTV | | |
| | | | SSGGGGSGGGGSGGGGS | | |
| | | | QIVLTQSPAIMSASPGE | | |
| | | | KVTMTCSASSSVSYMNW | | |
| | | | YQQKSGTSPKRWIYDTS | | |
| | | | KLASGVPAHFRGSGSGT | | |
| | | | SYSLTISGMEAEDAATY | | |
| | | | YCQQWSSNPFTFGSGTK | | |
| | | | LEIN (SEQ ID NO: 13) | | |
| PKSQ397: | EF1A | Mouse IGG heavy | OKT3ma scFv: | human CD8a aa 1-235: | Blasticidin |
| Mouse IgG signal | | chain: | MERHWIFLLLLSVTAGV | SHFVPVFLPAKPTTTPAP | |
| peptide, OKTlt lower | | MERHWIFLLLLS | HSQVQLQQSGAELARPG | RPPTPAPTIASQPLSLRP | |
| affinity scFv with | | VTAGVHS (SEQ | ASVKMSCKASGYTFTRY | EACRPAAGGAVHTRGLDF | |
| glycine-serine linker | | ID NO: 1) | TMHWVKQRPGQGLEWIG | ACDIYIWAPLAGTCGVLL | |
| fused to human CD8 | | | YINPSLGTTNYNQKFKD | LSLVITLYCNHRNRRRVC | |
| | | | KATLTTDKSSSTAYMQL | KCPRPVVKSGDKPSLSAR | |
| | | | SSLTSEDSAVYYCARYY | YV (SEQ ID NO: 12) | |
| | | | DDHYCLDYWGQGTTLTV | | |
| | | | SSGGGGSGGGGSGGGGS | | |
| | | | QIVLTQSPAIMSASPGE | | |
| | | | KVTMTCSASSSVSYMNW | | |
| | | | YQQKSGTSPKRWIYDTS | | |
| | | | KLASGVPAHFRGSGSGT | | |
| | | | SYSLTISGMEAEDAATY | | |
| | | | YCQQWSSNPFTFGSGTK | | |
| | | | LEIN (SEQ ID NO: 14) | | |

What is claimed is:

1. A method for assessing potency of tumor infiltrating lymphocytes (TILs), comprising:
coculturing TILs and immortalized cells, wherein the immortalized cells comprise a molecule that activates a T cell; and
assessing potency of the TILs.

2. The method of claim 1, wherein the molecule binds to a T cell antigen.

3. The method of claim 2, wherein the TILs express the T cell antigen.

4. The method of claim 2 or 3, wherein the T cell antigen is a CD3 antigen.

5. The method of any one of the preceding claims, wherein the immortalized cells express the molecule.

6. The method of any one of the preceding claims, wherein the molecule is an antibody or an antibody fragment.

7. The method of claim 6, wherein the antibody fragment is selected from a single-chain variable fragment (scFv), a F(ab′)2 fragment, a Fab fragment, a Fab′ fragment, and an Fv fragment.

8. The method of claim 7, wherein the antibody fragment is an scFv.

9. The method of any one of claims 6-8, wherein the antibody or antibody fragment is respectively an OKT3 antibody or OKT3 antibody fragment.

10. The method of claim 9, wherein the OKT3 antibody fragment is a membrane-bound OKT3 (mOKT3) scFv.

11. The method of claim 10, wherein the mOKT3 scFv is a low-affinity mOKT3 scFv variant.

12. The method of any one of claims 4-11, wherein the molecule binds to CD3 with a dissociation constant (KD) that is higher than the KD of mOKT3 scFv, wherein the KD of mOKT3 scFv is about 5× 10⁻¹⁰ M.

13. The method of claim 11 or 12, wherein the low-affinity mOKT3 scFv variant comprises an amino acid sequence having R55 and Y57 mutations, relative to the amino acid sequence of SEQ ID NO: 2.

14. The method of claim 13, wherein the low-affinity mOKT3 scFv variant comprises an amino acid sequence having R55M and Y57A mutations, relative to the amino acid sequence of SEQ ID NO: 2.

15. The method of claim 14, wherein the low-affinity mOKT3 scFv variant binds to CD3 with a dissociation constant KD that is least 250-fold higher than the KD of mOKT3 scFv.

16. The method of claim 13, wherein the low-affinity mOKT3 scFv variant comprises an amino acid sequence having R55L and Y57T, relative to the amino acid sequence of SEQ ID NO: 2.

17. The method of claim 16, wherein the low-affinity mOKT3 scFv variant binds to CD3 with a dissociation constant KD that is least 1000-fold higher than the KD of OKT3 scFv.

18. The method of any one of claims 1-5, wherein the molecule is selected from bacterial superantigens, optionally Staphylococcal enterotoxin B (SEB), phytohaemagglutinin (PHA) and concanavalin A (ConA).

19. The method of any one of the preceding claims, wherein the molecule is a membrane-tethered molecule.

20. The method of any one of the preceding claims, wherein the TILs are engineered TILs (eTILs).

21. The method of claim 20, wherein the eTILs are edited eTILs.

22. The method of claim 21, wherein the edited eTILs comprise a genomic modification.

23. The method of any one of the preceding claims, wherein the immortalized cells comprise a clonal population of immortalized cells.

24. The method of any one of the preceding claims, wherein the immortalized cells are immortalized human cells.

25. The method of any one of the preceding claims, wherein the immortalized cells are engineered cancer cells.

26. The method of any one of the preceding claims, wherein the engineered cancer cells are selected from engineered melanoma cells, engineered colorectal cancer cells, engineered bile duct cancer cells, and engineered breast cancer cells.

27. The method of any one of the preceding claims, wherein prior to the coculturing the immortalized cells are plated on an ultra-low attachment surface.

28. The method of any one of the preceding claims, wherein prior to the coculturing, the immortalized cells are plated in a multiwell plate, optionally a 6-well, 24-well, or 96-well plate.

29. The method of claim 27 or 28, wherein the immortalized cells are plated at density of about 5,000 to about 10,000 live cells/culture.

30. The method of any one of claims 27-29, wherein the ultra-low attachment surface comprises a hydrophilic, neutrally charged hydrogel coating.

31. The method of any one of claims 27-30, wherein the immortalized cells are cultured, preferably for about 2 to 6 days, more preferably about 4 days, to produce three-dimensional spheroids.

32. The method of any one of the preceding claims, wherein prior to the coculturing, the TILs were added to a culture of the immortalized cells at an ET ratio of about 20:1 to 1:1.

33. The method of any one of the preceding claims, wherein the coculturing is for about 4 hours, about 8 hours, about 12 hours, about 24 hours, about 36 hours, about 48 hours, about 3 days, about 4 days, or about 5 days, optionally wherein the coculturing is for about 1 day to about 5 days.

34. The method of any one of the preceding claims, wherein the assessing potency comprises measuring cytokine release, optionally IFN-γ, IL-2, TNFα and/or IL-6 release, from the immortalized cells, cell death and/or viability of the immortalized cells.

35. The method of any one of the preceding claims, wherein the assessing potency comprises measuring growth, cell death, and/or viability of the immortalized cells.

36. The method of any one of the preceding claims, wherein the measuring comprises performing an assay selected from real-time cell viability assays, ATP cell viability assays, live cell protease viability assays, tetrazolium reduction cell viability assays, resazurin reduction cell viability assays, dead-cell protease release cytotoxicity assays, lactate dehydrogenase release cytotoxicity assays, and DNA dye cytotoxicity assays.

37. A method for assessing potency of tumor infiltrating lymphocytes (TILs), comprising:
coculturing TILs and a clonal population of engineered cancer cells, wherein the engineered cancer cells express an anti-CD3 antibody or anti-CD3 antibody fragment; and
assessing death and/or viability of the engineered cancer cells.

38. The method of claim 37, wherein the TILs express a CD3 antigen.

39. The method of claim 37 or 38, wherein the engineered cancer cells express an anti-CD3 antibody fragment.

40. The method of claim 39, wherein the anti-CD3 antibody fragment is an anti-CD3 single-chain variable fragment (scFv).

41. The method of claim 40, wherein the anti-CD3 scFv is mOKT3 scFv.

42. The method of claim 41, wherein the mOKT3 scFv is a low-affinity mOKT3 scFv variant.

43. The method of claim 42, wherein the low-affinity mOKT3 scFv variant binds to CD3 with a dissociation constant (KD) that is lower than the KD of mOKT3 scFv, wherein the KD of mOKT3 scFv is about 5×10⁻¹⁰ M.

44. The method of claim 43, wherein the low-affinity mOKT3 scFv variant binds to CD3 with a dissociation constant KD that is least 1000-fold lower than the KD of mOKT3 scFv.

45. The method of claim 43 or 44, wherein the low-affinity mOKT3 scFv variant comprises an amino acid sequence having R55L and Y57T mutations, relative to the amino acid sequence of SEQ ID NO: 2.

46. The method of any one of claims 37-45, wherein the anti-CD3 antibody or anti-CD3 antibody fragment is respectively a membrane-tethered anti-CD3 antibody or membrane-tethered anti-CD3 antibody fragment.

47. The method of any one of claims 37-46, wherein the TILs are engineered TILs (eTILs).

48. The method of claim 47, wherein the eTILs are edited eTILs.

49. The method of claim 48, wherein the edited eTILs comprise a genomic modification.

50. The method of any one of claims 37-49, wherein the engineered cancer cells are selected from engineered melanoma cells, engineered colorectal cancer cells, engineered bile duct cancer cells, and engineered breast cancer cells.

51. The method of claim 50, wherein the engineered cancer cells are engineered melanoma cells.

52. The method of any one of claims 37-51, wherein the coculturing is for at least 24 hours, optionally about 24 to 72 hours.

53. The method of any one of claims 37-52, wherein the measuring comprises performing an assay selected from real-time cell viability assays, ATP cell viability assays, live cell protease viability assays, tetrazolium reduction cell viability assays, resazurin reduction cell viability assays, dead-cell protease release cytotoxicity assays, lactate dehydrogenase release cytotoxicity assays, and DNA dye cytotoxicity assays.

54. A method for assessing potency of tumor infiltrating lymphocytes (TILs), comprising:
coculturing TILs, immortalized cells, and a bispecific molecule that activates a T cell and binds to the immortalized cells; and
assessing potency of the TILs.

55. The method of claim 54, wherein the TILs express a CD3 antigen.

56. The method of claim 54 or 55, wherein the bispecific molecule comprises a molecule that binds CD3.

57. The method of any one of claims 54-56, wherein the TILs are engineered TILs (eTILs).

58. The method of claim 57, wherein the eTILs are edited eTILs.

59. The method of claim 58, wherein the edited eTILs comprise a genomic modification.

60. The method of any one of claims 54-59, wherein the immortalized cells comprise a clonal population of immortalized cells.

61. The method of any one of claims 54-60, wherein the immortalized cells are human cells.

62. The method of any one of claims 54-61, wherein the immortalized cells are cancer cells.

63. The method of claim 62, wherein the cancer cells are selected from melanoma cells, colorectal cancer cells, bile duct cancer cells, and breast cancer cells.

64. The method of claim 62 or 63, wherein the bispecific molecule comprises a molecule that binds CD19.

65. The method of claim 64, wherein the bispecific molecule is a CD19-CD3 BITER.

66. The method of any one of claims 54-65, wherein the coculturing is for at least 24 hours, optionally about 24 to 72 hours.

67. The method of any one of claims 54-66, wherein the measuring comprises performing an assay selected from real-time cell viability assays, ATP cell viability assays, live cell protease viability assays, tetrazolium reduction cell viability assays, resazurin reduction cell viability assays, dead-cell protease release cytotoxicity assays, lactate dehydrogenase release cytotoxicity assays, and DNA dye cytotoxicity assay.

68. A method for assessing potency of polyclonal T cells, comprising:
coculturing polyclonal T cells and immortalized cells, wherein the immortalized cells comprise a molecule that activates a T cell; and
assessing potency of the polyclonal T cells.

69. The method of claim 68, wherein the molecule binds to a T cell antigen.

70. The method of claim 69, wherein the polyclonal T cells express the T cell antigen.

71. The method of claim 69 or 70, wherein the T cell antigen is a CD3 antigen.

72. The method of any one of the preceding claims, wherein the immortalized cells express the molecule.

73. The method of any one of the preceding claims, wherein the molecule is an antibody or an antibody fragment.

74. The method of claim 73, wherein the antibody fragment is selected from a single-chain variable fragment (scFv), a F(ab′)2 fragment, a Fab fragment, a Fab′ fragment, and an Fv fragment.

75. The method of claim 74, wherein the antibody fragment is an scFv.

76. The method of any one of claims 73-75, wherein the antibody or antibody fragment is respectively an OKT3 antibody or OKT3 antibody fragment.

77. The method of claim 76, wherein the OKT3 antibody fragment is a membrane-bound OKT3 (mOKT3) scFv.

78. The method of claim 77, wherein the mOKT3 scFv is a low-affinity mOKT3 scFv variant.

79. The method of any one of claims 71-78, wherein the molecule binds to CD3 with a dissociation constant (KD) that is higher than the KD of mOKT3 scFv, wherein the KD of mOKT3 scFv is about 5×10⁻¹⁰ M.

80. The method of claim 78 or 79, wherein the low-affinity mOKT3 scFv variant comprises an amino acid sequence having R55 and Y57 mutations, relative to the amino acid sequence of SEQ ID NO: 2.

81. The method of claim 80, wherein the low-affinity mOKT3 scFv variant comprises an amino acid sequence having R55M and Y57A mutations, relative to the amino acid sequence of SEQ ID NO: 2.

82. The method of claim 81, wherein the low-affinity mOKT3 scFv variant binds to CD3 with a dissociation constant KD that is least 250-fold higher than the KD of mOKT3 scFv.

83. The method of claim 74, wherein the low-affinity mOKT3 scFv variant comprises an amino acid sequence having R55L and Y57T, relative to the amino acid sequence of SEQ ID NO: 2.

84. The method of claim 83, wherein the low-affinity mOKT3 scFv variant binds to CD3 with a dissociation constant KD that is least 1000-fold higher than the KD of OKT3 scFv.

85. The method of any one of claims 68-72, wherein the molecule is selected from bacterial superantigens, optionally Staphylococcal enterotoxin B (SEB), phytohaemagglutinin (PHA) and concanavalin A (ConA).

86. The method of any one of the preceding claims, wherein the molecule is a membrane-tethered molecule.

87. The method of claim 68, wherein the polyclonal T cells comprise neoantigen-specific T cells.

88. The method of claim 68, wherein the polyclonal T cells are from peripheral blood.

89. The method of claim 68, wherein the polyclonal T cells are from bone marrow.

90. The method of any one of the preceding claims, wherein the immortalized cells comprise a clonal population of immortalized cells.

91. The method of any one of the preceding claims, wherein the immortalized cells are immortalized human cells.

92. The method of any one of the preceding claims, wherein the immortalized cells are engineered cancer cells.

93. The method of any one of the preceding claims, wherein the engineered cancer cells are selected from engineered melanoma cells, engineered colorectal cancer cells, engineered bile duct cancer cells, and engineered breast cancer cells.

94. The method of any one of the preceding claims, wherein the coculturing is for at least 4 hours.

95. The method of any one of the preceding claims, wherein the assessing potency comprises measuring cytokine release, optionally IFN-γ and/or IL-6 release, from the immortalized cells, cell death and/or viability of the immortalized cells.

96. The method of any one of the preceding claims, wherein the assessing potency comprises measuring growth, cell death, and/or viability of the immortalized cells.

97. The method of any one of the preceding claims, wherein the measuring comprises performing an assay selected from real-time cell viability assays, ATP cell viability assays, live cell protease viability assays, tetrazolium reduction cell viability assays, resazurin reduction cell viability assays, dead-cell protease release cytotoxicity assays, lactate dehydrogenase release cytotoxicity assays, and DNA dye cytotoxicity assays.

98. A method for assessing potency of polyclonal T cells, comprising:
coculturing polyclonal T cells, immortalized cells, and a bispecific molecule that activates a T cell and binds to the immortalized cells; and
assessing potency of the polyclonal T cells.

99. The method of claim 98, wherein the polyclonal T cells express a CD3 antigen.

100. The method of claim 98 or 99, wherein the bispecific molecule comprises a molecule that binds CD3.

101. The method of claim 98, wherein the polyclonal T cells comprise neoantigen-specific T cells.

102. The method of claim 99, wherein the polyclonal T cells are from peripheral blood.

103. The method of claim 99, wherein the polyclonal T cells are from bone marrow.

104. The method of any one of claims 98-102, wherein the immortalized cells comprise a clonal population of immortalized cells.

105. The method of any one of claims 98-104, wherein the immortalized cells are human cells.

106. The method of any one of claims 98-105, wherein the immortalized cells are cancer cells.

107. The method of claim 106, wherein the cancer cells are selected from melanoma cells, colorectal cancer cells, bile duct cancer cells, and breast cancer cells.

108. The method of claim 106 or 107, wherein the bispecific molecule comprises a molecule that binds CD19.

109. The method of claim 108, wherein the bispecific molecule is a CD19-CD3 BITE®.

110. The method of any one of claims 98-109, wherein the coculturing is for at least 24 hours, optionally about 24 to 72 hours.

111. The method of any one of claims 98-110, wherein the measuring comprises performing an assay selected from real-time cell viability assays, ATP cell viability assays, live cell protease viability assays, tetrazolium reduction cell viability assays, resazurin reduction cell viability assays, dead-cell protease release cytotoxicity assays, lactate dehydrogenase release cytotoxicity assays, and DNA dye cytotoxicity assays.


SEQUENCE LISTING (20 sequences)

SEQ ID NO: 1 - protein, 19 aa, Mus musculus
MERHWIFLLLLSVTAGVHS

SEQ ID NO: 2 - protein, 240 aa, Mus musculus
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIN

SEQ ID NO: 3 - protein, 108 aa, Mus musculus
SSVVPVLQKVNSTTTKPVLRTPSPVHPTGTSQPQRPEDCRPRGSVKGTGLDFACDIYIWAPLAGICVALLLSLIITLICYHRSRKRVCKCPRPLVRQEGKPRPSEKIV

SEQ ID NO: 4 - protein, 22 aa, Homo sapiens
MLLLVTSLLLCELPHPAFLLIP

SEQ ID NO: 5 - protein, 244 aa, synthetic construct
QVQLLESGAELARPGASVKMSCKASGYTFTRYTMHWVKQRPGQGLEWIGYINPSRGYTNYNQKFKDKATLTTDKSSSTAYMQLSSLTSEDSAVYYCAGYYDDHYCLDYWGQGTLVTVSSGGGGSGGGGSGGGGSDIVMTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIKRTTT

SEQ ID NO: 6 - protein, 99 aa, Homo sapiens
TTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVN

SEQ ID NO: 7 - protein, 243 aa, synthetic construct
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAKTTPKLEEGEFSEARVDIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 8 - protein, 21 aa, Homo sapiens
MPMGSLQPLATLYLLGMLVAS

SEQ ID NO: 9 - protein, 249 aa, synthetic construct
VLAQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTVTVSAGGGGSGGGGSGGGSSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRGGGDP

SEQ ID NO: 10 - protein, 79 aa, Homo sapiens
PSPLFPGPSKPFWVLVVVGGVLACYSLLVTVAFIIFWVRSKRSRLLHSDYMNMTPRRPGPTRKHYQPYAPPRDFAAYRS

SEQ ID NO: 11 - protein, 356 aa, Homo sapiens
TTPEPCELDDEDFRCVCNFSEPQPDWSEAFQCVSAVEVEIHAGGLNLEPFLKRVDADADPRQYADTVKALRVRRLTVGAAQVPAQLLVGALRVLAYSRLKELTLEDLKITGTMPPLPLEATGLALSSLRLRNVSWATGRSWLAELQQWLKPGLKVLSIAQAHSPAFSCEQVRAFPALTSLDLSDNPGLGERGLMAALCPHKFPAIQNLALRNTGMETPTGVCAALAAAGVQPHSLDLSHNSLRATVNPSAPRCMWSSALNSLNLSFAGLEQVPKGLPAKLRVLDLSCNRLNRAPQPDELPEVDNLTLDGNPFLVPGTALPHEGSMNSGVVPACARSTLSVGVSGTLVLLQGARGFA

SEQ ID NO: 12 - protein, 110 aa, Homo sapiens
SHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 13 - protein, 259 aa, synthetic construct
MERHWIFLLLLSVTAGVHSQVQLQQSGAELARPGASVKMSCKASGYTFTRYTMHWVKQRPGQGLEWIGYINPSMGATNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIN

SEQ ID NO: 14 - protein, 259 aa, synthetic construct
MERHWIFLLLLSVTAGVHSQVQLQQSGAELARPGASVKMSCKASGYTFTRYTMHWVKQRPGQGLEWIGYINPSLGTTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIN

SEQ ID NO: 15 - protein, 18 aa, synthetic construct
EGRGSLLTCGDVEENPGP

SEQ ID NO: 16 - protein, 132 aa, synthetic construct
MAKPLSQEESTLIERATATINSIPISEDYSVASAALSSDGRIFTGVNVYHFTGGPCAELVVLGTAAAAAAGNLTCIVAIGNENRGILSPCGRCRQVLLDLHPGIKAIVKDSDGQPTAVGIRELLPSGYVWEG

SEQ ID NO: 17 - protein, 213 aa, Mus musculus
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRADTAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 18 - protein, 450 aa, Mus musculus
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAKTTAPSVYPLAPVCGGTTGSSVTLGCLVKGYFPEPVTLTWNSGSLSSGVHTFPAVLQSDLYTLSSSVTVTSSTWPSQSITCNVAHPASSTKVDKKIEPRPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 20 - protein, 351 aa, Homo sapiens
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN